(19) United States
(12) Patent Application Publication
(10) Pub. No.: US 2026/0242327 A1
(43) Pub. Date: Aug. 20, 2026

(54) INTERMOLECULAR CYCLOPROPANATION OF UNSATURATED HYDROCARBONS

(71) Applicant: THE PENN STATE RESEARCH FOUNDATION, University Park, PA (US)

(72) Inventors: Ramesh GIRI, University Park, PA (US); Dhruba POUDEL, University Park, PA (US)

(21) Appl. No.: 19/161,542

(22) PCT Filed: Mar. 1, 2024
(86) PCT No.: PCT/US2024/018150

§ 371 Date: Sep. 2, 2025

Related U.S. Application Data
(60) Provisional application No. 63/488,046, filed on Mar. 2, 2023.
(60) Provisional application No. 63/517,257, filed on Aug. 2, 2023.

(51) Int. Cl. C07C 67/343; B01J 31/02; B01J 31/18; B01J 31/22; B01J 35/39; C07B 37/10; C07D 213/55; C07D 307/54; C07D 333/24

(52) U.S. Cl. CPC C07C 67/343; B01J 31/0244; B01J 31/1815; B01J 31/2295; B01J 35/39; C07B 37/10; C07D 213/55; C07D 307/54; C07D 333/24; B01J 2231/325; B01J 2531/004; B01J 2531/827; C07C 2601/02

(57) ABSTRACT

Disclosed are methods for forming a cyclopropyl or cyclopropenyl ring in a compound by, for example, contacting an unsaturated hydrocarbon and an active methylene compound with an oxidant and a catalytically effective amount of a photocatalyst; and irradiating the photocatalyst with a light source to form a cyclopropyl or cyclopropenyl-containing product. Also described herein are methods for forming a cyclopropyl or cyclopropenyl ring in a compound by contacting an unsaturated hydrocarbon and an active methylene compound with an oxidant under conditions effective to form a cyclopropyl or cyclopropenyl ring. Additionally disclosed herein are compounds comprising a cyclopropyl or cyclopropenyl ring formed by the methods disclosed herewith.

### CROSS-REFERENCE TO RELATED APPLICATIONS

This application claims benefit of priority of U.S. Provisional Application No. 63/488,046, filed Mar. 2, 2023, and U.S. Provisional Application No. 63/517,257, filed Aug. 2, 2023, each of which is hereby incorporated by reference in its entirety.

### STATEMENT REGARDING FEDERALLY SPONSORED RESEARCH OR DEVELOPMENT

This invention was made with government support under Grant No. R35GM133438 awarded by the National Institutes of Health. The Government has certain rights in the invention.

### BACKGROUND

Cyclopropanes are highly valuable and versatile intermediates in the synthesis of complex molecules and natural products. Cyclopropyl moieties are also extensively featured as a pivotal design element in preclinical and clinical drug molecules to achieve specific therapeutic goals, and a key motif in biologically active molecules and natural products. For example, the recently FDA-approved drugs, Nirmatrelvir (Paxlovid™) for Covid-19, Mitapivat (Pyrukynd) for hemolytic anemia, Deucravacitinib (Sotyktu) for plaque psoriasis, and 14 out of 200 top-selling small molecule commercial drugs, including Singulair (asthma), Telaprevir (Hepatitis C) and Abacavir (HIV/AIDS), contain a cyclopropyl ring as a key element. Cyclopropanation is therefore one of the most heavily investigated strained-ring forming reactions in organic synthesis. Yet, the carbonaceous triangle is also among the most difficult rings to create because of the strain, which destabilizes the ring (by 17 kcal/mol) and renders it prone to opening during reactions.

Among the limited methods available for intermolecular cyclopropanation, the most utilized and reliable method is carbene insertion into alkenes. This method is profoundly dependent on reactive carbene and metal-carbenoid intermediates [[M]=CR₂], in particular the donor-acceptor Rh-carbenoids, derived from highly energetic diazoalkanes, which require stringent precautions for handling. The metal-carbenoids are typically generated from diazomethane (CH₂N₂), TMSCHN₂, PhCHN₂, and α-diazocarbonyl compounds by decomposition in reaction with transition-metal [M] catalysts, and more recently under photoredox and engineered enzymatic conditions. The Simmons-Smith and related reactions also transform alkenes into cyclopropanes, yet again, these reactions require a large excess of unstable, often difficult-to-access 1,1- and 1,2-dihaloalkanes and highly reactive Et₂Zn or Zn/Cu reagents to generate an adequate quantity of zinc carbenoids for satisfactory reactivity.

Recently, catalytic methods have emerged to simplify cyclopropanation of alkenes with alternate precursors. However, these processes also pivot on the generation and exploitation of [M]=CR₂ intermediates, and multistep synthetic manipulations to arrive at reactive [M]=CR₂ intermediates in situ. Alternatively, the non-carbenoid approaches, such as the Johnson-Corey-Chaykovsky reaction via a Michael-addition initiated ring closure require careful molecular prefunctionalization to install a leaving group at a precise reaction site either on Michael acceptors (α,β-unsaturated carbonyls) or Michael donors, as well as a strong base, such as LDA, KHMDS, NaNH₂ or BuLi, to enolize the Michael donors for sufficient nucleophilicity. Additionally, the Kulinkovick reaction, which converts terminal alkenes into cyclopropyl alcohols and amines, suffers fundamentally from functional group incompatibility and 1,2-addition reactions owing to the use of superstoichiometric quantities of titanium salts and alkyl Grignard reagents to generate titanacyclopropanes in situ as cyclopropanating reagents. Thus, the catalytic intermolecular construction of cyclopropyl rings on unactivated alkenes conveniently with simple, readily available and bench-stable chemicals that in operationally straightforward and broadly applicable fashion is an ongoing challenge.

### SUMMARY

Disclosed herein are compounds, compositions, methods for making and using such compounds and compositions. In one aspect, disclosed herein is a photoredox approach for the intermolecular cyclopropanation of unactivated alkenes directly with active methylene compounds through cooperativity of photosensitized dioxygen (O₂) and alkyl halide (e.g., alkyl iodide) via an electron relay system. The disclosed methods can offer a convenient protocol with bench-stable chemicals under neutral reaction conditions in air/O₂ without requiring special safety and sensitivity precautions. In addition, since hundreds of active methylene compounds with varied functional substituents are commercially available and a large number of their analogs can be synthesized and stored for prolonged periods for use on demand, the current reaction can provide a simple, practical, and broad-scope method for the intermolecular cyclopropanation of unactivated alkenes.

In various aspects, disclosed herein are methods for forming a cyclopropyl or cyclopropenyl ring in a compound comprising contacting an unsaturated hydrocarbon and an active methylene compound with an oxidant and a catalytically effective amount of a photocatalyst; and irradiating the photocatalyst with a light source to form a cyclopropyl or cyclopropenyl-containing product.

In some examples, the unsaturated hydrocarbon comprises an alkene. In some examples, the alkene comprises a terminal alkene. In some examples, the terminal alkene comprises an alkene defined by the formula R¹(CH)CH₂,
wherein
R¹ is hydrogen, a halogen, a substituted or unsubstituted aryl, a substituted or unsubstituted alkyl, a substituted or unsubstituted alkylaryl, a substituted or unsubstituted heteroaryl, a substituted or unsubstituted alkylheteroaryl, —CHO, —CO₂R^{a}, —N(R^{a})₂, or —OR^{a}; and
each R^{a}, when present, is independently a substituted or unsubstituted aryl, a substituted or unsubstituted alkyl, a substituted or unsubstituted alkylaryl, a substituted or unsubstituted heteroaryl, or a substituted or unsubstituted alkylheteroaryl. In some examples, the terminal alkene comprises 3-butenylbenzene. In some examples, the alkene comprises an internal alkene. In some examples, the unsaturated hydrocarbon comprises an alkyne.

In some examples, the active methylene compound is defined by the formula R² (CH₂) R³, wherein R² and R³ are each independently an electron withdrawing group. In some examples, R² and R³ are each independently an electron withdrawing group selected from the group consisting of a halogen, —C(O)R^{b}, —COOR^{b}, —C(O)NR^{b}R^{c}, —CN, —SO₃R^{b}, —SO₂R^{b}, —SO₂NR^{b}R^{c}, —OP(O)OR^{b}OR^{c}, —R^{b}NCO, and —NO₂, wherein R^{b} and R^{c} when present, are independently H or a substituted or unsubstituted C₁-C₆ alkyl. In some examples, the active methylene compound is defined by the formula R^{b}OOC(CH₂)COOR^{c}, wherein R^{b} and R^{c} are independently H or a substituted or unsubstituted C₁-C₆ alkyl. In some examples, the active methylene compound comprises diethylmalonate.

In some examples, the oxidant comprises a peroxide. In some examples, the oxidant comprises an alkyl halide. In some examples, the oxidant comprises an alkyl iodide (e.g., a cycloalkyl iodide). In some examples, the alkyl iodide comprises iodocyclohexane. In some examples, the oxidant comprises a first oxidant and a second oxidant. In some examples, the first oxidant comprises an alkyl halide. In some examples, the second oxidant comprises O₂ (e.g., air). In some examples, the second oxidant comprises a peroxide.

In some examples, the photocatalyst comprises an organic or organometallic photocatalyst. In some examples, the photocatalyst comprises 4CzIPN, Eosin Y salts, [Ir(dtbbpy)(ppy)₂][BF₄], [Ir(dFCF₃ppy))₂ (bpy)]PF₆, Ir(ppy)₃, or a combination thereof. In some examples, the photocatalyst is irradiated with visible light. In some examples, the visible light has a wavelength of from 390 nanometers (nm) to 467 nm.

In some examples, the unsaturated hydrocarbon, active methylene compound, oxidant, and photocatalyst are contacted in a solvent. In some examples, the solvent comprises dimethylformamide (DMF), dioxane, dimethylacetamide (DMA), dimethyl sulfoxide (DMSO), N-methyl-2-pyrrolidone (NMP), toluene, dichloromethane, or a combination thereof. In some examples, the solvent comprises DMF.

In some examples, the method performed at ambient temperatures (e.g., from 10° C. to 50° C., from 10° C. to 40° C., from 10° C. to 30° C., from 20° C. to 50° C., from 30° C. to 50° C., or from 30° C. to 40° C.).

In some examples, a yield of the cyclopropyl or cyclopropenyl-containing product is 30% or more, such as 40% or more, 50% or more, 60% or more, 70% or more, 80% or more, 90% or more, 95% or more, or 99% or more.

In some examples, the method is used to form the cyclopropyl or cyclopropenyl ring in any of the compounds shown in the Examples.

Also described herein are methods for forming a cyclopropyl or cyclopropenyl ring in a compound by contacting an unsaturated hydrocarbon and an active methylene compound with an oxidant under conditions effective to form a cyclopropyl or cyclopropenyl ring.

In some examples, the unsaturated hydrocarbon comprises an alkene. In some examples, the alkene comprises a terminal alkene. In some examples, the terminal alkene comprises an alkene defined by the formula R¹ (CH)CH₂, wherein
R¹ is hydrogen, a halogen, a substituted or unsubstituted aryl, a substituted or unsubstituted alkyl, a substituted or unsubstituted alkylaryl, a substituted or unsubstituted heteroaryl, a substituted or unsubstituted alkylheteroaryl, —CHO, —CO₂R^{a}, —N(R^{a})₂, or —OR^{a}; and
each R^{a}, when present, is independently a substituted or unsubstituted aryl, a substituted or unsubstituted alkyl, a substituted or unsubstituted alkylaryl, a substituted or unsubstituted heteroaryl, or a substituted or unsubstituted alkylheteroaryl. In some examples, the terminal alkene comprises 3-butenylbenzene. In some examples, the alkene comprises an internal alkene.

In some examples, the unsaturated hydrocarbon comprises an alkyne.

In some examples, the active methylene compound is defined by the formula R³ (CH₂) R⁴, wherein R³ and R⁴ are each independently an electron withdrawing group.

In some examples, R² and R³ are each independently an electron withdrawing group selected from the group consisting of a halogen, —C(O)R^{b}, —COOR^{b}, —C(O)NR^{b}R^{c}, —CN, —SO₃R^{b}, —SO₂R^{b}, —SO₂NR^{b}R^{c}, —OP(O)OR^{b}OR^{c}, —R^{b}NCO, and —NO₂, wherein R^{b} and R^{c}, when present, are independently H or a substituted or unsubstituted C₁-C₆ alkyl. In some examples, the active methylene compound is defined by the formula R^{b}OOC(CH₂)COOR^{c}, wherein R^{b} and R^{c} are independently H or a substituted or unsubstituted C₁-C₆ alkyl. In some examples, the active methylene compound comprises diethylmalonate.

In some examples, the oxidant comprises a peroxide. In some examples, the oxidant comprises an alkyl halide. In some examples, the oxidant comprises an alkyl iodide (e.g., a cycloalkyl iodide). In some examples, the alkyl iodide comprises iodocyclohexane.

In some examples, the oxidant comprises a first oxidant and a second oxidant. In some examples, the first oxidant comprises an alkyl halide. In some examples, the second oxidant comprises O₂ (e.g., air). In some examples, the second oxidant comprises a peroxide. In some examples, the oxidant comprises NaIO₄. In some examples, the oxidant comprises MnO₂.

In some examples, the unsaturated hydrocarbon, active methylene compound, and oxidant are contacted in a solvent. In some examples, the solvent comprises dimethylformamide (DMF), dioxane, dimethylacetamide (DMA), dimethyl sulfoxide (DMSO), N-methyl-2-pyrrolidone (NMP), toluene, dichloromethane, or a combination thereof. In some examples, the solvent comprises DMF.

In some examples, the method is performed at ambient temperatures (e.g., from 10° C. to 50° C., from 10° C. to 40° C., from 10° C. to 30° C., from 20° C. to 50° C., from 30° C. to 50° C., or from 30° C. to 40° C.).

In some examples, a yield of the cyclopropyl or cyclopropenyl-containing product is 30% or more, such as 40% or more, 50% or more, 60% or more, 70% or more, 80% or more, 90% or more, 95% or more, or 99% or more.

Additionally disclosed herein are compounds comprising a cyclopropyl or cyclopropenyl ring formed by the methods disclosed herein.

Additional advantages of the disclosed subject matter will be set forth in part in the description that follows and the Figures, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying figures, which are incorporated in and constitute a part of this specification, illustrate several aspects of the disclosure, and together with the description, serve to explain the principles of the disclosure.

FIG. 1 shows several pharmaceutical compounds containing a cyclopropyl ring that can be synthetized using the methods described herein.

FIG. 2 shows a comparison between existing strategies for intermolecular alkene cyclopropanation and intermolecular cyclopropanation of unactivated alkenes directly with active methylene compounds through an O₂/alkyl halide electron relay pair system.

FIG. 3 shows reaction variations of the intermolecular cyclopropanation of unactivated alkenes directly with active methylene compounds. Reactions were conducted in 0.10 mmol scale, unless stated otherwise, in 0.50 mL DMF at 440 nm blue LED (36 Watt Kessil lamp at 100% intensity) and ambient temperature (~35° C. controlled by fans) in 1 dram capped glass vials. Yields were determined by GC with trimethoxybenzene as a standard. 3 h in O₂ and 16 h in air.

FIG. 4 shows several example cyclopropyl-containing compounds synthesized by reacting 3-butenylbenzene with different active methylene compounds according to the presently disclosed methods. Reactions were conducted in 1.0 mmol scale, unless stated otherwise, in 5.0 mL DMF at 440 nm blue LED (36 Watt Kessil lamp at 100% intensity) and ambient temperature (~35-40° C. controlled by fans) under O₂ in 6 dram capped glass vials. Reported yields are for isolated products. dr was determined by ¹H NMR.

FIG. 5 shows several example cyclopropyl-containing compounds synthesized by reacting different unactivated alkenes with diethyl malonate according to the presently disclosed methods. Reactions were conducted in 1.0 mmol scale, unless stated otherwise, in 5.0 mL DMF at 440 nm blue LED (36 Watt Kessil lamp at 100% intensity) and ambient temperature (~35-40° C. controlled by fans) under O₂ in 6 dram capped glass vials. Reported yields are for isolated products. dr was determined by ¹H NMR.

FIG. 6 shows example cyclopropyl-containing drugs and natural products synthesized according to an aspect of the present disclosure. Reactions were conducted in 1.0 mmol scale, unless stated otherwise, in 5.0 mL DMF at 440 nm blue LED (36 Watt Kessil lamp at 100% intensity) and ambient temperature (~35-40° C. controlled by fans) under O₂ in 6 dram capped glass vials. Reported yields are for isolated products. dr was determined by ¹H NMR.

FIG. 7 shows several example cyclopropyl-containing compounds synthesized by reacting butene gas with different active methylene compounds according to the presently disclosed methods. Reactions were conducted in 1.0 mmol scale, unless stated otherwise, in 5.0 mL DMF at 440 nm blue LED (36 Watt Kessil lamp at 100% intensity) and ambient temperature (~35-40° C. controlled by fans) under O₂ in 6 dram capped glass vials. Reported yields are for isolated products. dr was determined by ¹H NMR.

FIG. 8 shows several example cyclopropyl-containing compounds synthesized by reacting propylene gas with different active methylene compounds according to the presently disclosed methods. Reactions were conducted in 1.0 mmol scale, unless stated otherwise, in 5.0 mL DMF at 440 nm blue LED (36 Watt Kessil lamp at 100% intensity) and ambient temperature (~35-40° C. controlled by fans) under O₂ in 6 dram capped glass vials. Reported yields are for isolated products. dr was determined by ¹H NMR.

FIG. 9 shows several example cyclopropyl-containing compounds synthesized by reacting ethylene gas with different active methylene compounds according to the presently disclosed methods. Reactions were conducted in 1.0 mmol scale, unless stated otherwise, in 5.0 mL DMF at 440 nm blue LED (36 Watt Kessil lamp at 100% intensity) and ambient temperature (~35-40° C. controlled by fans) under O₂ in 6 dram capped glass vials. Reported yields are for isolated products. dr was determined by ¹H NMR.

FIG. 10 shows several example cyclopropyl-containing compounds synthesized by reacting unactivated alkenes with different active methylene compounds according to the presently disclosed methods. Reactions were conducted in 1.0 mmol scale, unless stated otherwise, in 5.0 mL DMF at 440 nm blue LED (36 Watt Kessil lamp at 100% intensity) and ambient temperature (~35-40° C. controlled by fans) with NaIO₄ as an oxidant in 6 dram capped glass vials. Reported yields are for isolated products. dr was determined by ¹H NMR.

FIG. 11 shows an illustrated mechanism of an example catalytic cycle by iodine generation and detection, product profile and light on-off experiment.

FIG. 12 shows a catalytic reaction with I₂, and further mechanistic studies probing the potential involvement of carbenes and α-iodocarbonyls as reaction intermediates.

### DETAILED DESCRIPTION

The materials, compounds, compositions, articles, and methods described herein may be understood more readily by reference to the following detailed description of specific aspects of the disclosed subject matter and the Examples and Figures included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “an agent” includes mixture of two or more such agents, and the like.

“Optional” or “optionally” means that the subsequently described event or circumstance can or cannot occur, and that the description includes instances where the event or circumstance occurs and instances where it does not.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

As used herein, the term “alkene” refers to a straight chain or branched hydrocarbon having at least 2 carbon atoms and at least one double bond. A “terminal alkene” refers to an alkene wherein a double bond is between two carbon atoms at the end of the hydrocarbon chain. An “internal alkene” refers to an alkene wherein a double bond is between two carbon atoms that are not at the end of the hydrocarbon chain. The terms are not intended to be mutually exclusive and an alkene compound can effectively be classified as both a “terminal” and “internal” alkene.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula-C(O)H. Throughout this specification “C(O)” or “CO” is a short hand notation for C═O, which is also referred to herein as a “carbonyl.”

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “carbamide” means compounds having the group —N(Z¹)—(CO)N(Z¹)₂ where each Z¹ can be, independently, an alkyl, alkenyl, alkynyl, aryl, arylalkyl, cycloalkyl, carbonyl, ether, haloalkyl, heteroaryl and heterocyclyl.

The term “carbamate” means a group of the form —Z¹OC(O)N(Z¹)—, —Z¹⁰C(O)N(Z¹)Z¹—, or —OC(O)N(Z¹)₂, where each Z¹ can be, independently, an alkoxy, aryloxy, alkyl, alkenyl, alkynyl, aryl, arylalkyl, cycloalkyl, ether, formyl, haloalkyl, heteroaryl, and heterocyclyl. Carbamates include, e.g., arylcarbamates and heteroaryl carbamates.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “formamide” refers to compounds comprising the —NC(O)H formamide group. Formamides include compounds having the formula HC(O)NZ¹Z² wherein Z¹ and Z² can be, independently, hydrogen or an alkyl, alkenyl, alkynyl, aryl, arylalkyl, cycloalkyl, carbonyl, ether, haloalkyl, heteroaryl and heterocyclyl.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine.

The term “hydroxyl” as used herein is represented by the formula-OH.

The term “nitro” as used herein is represented by the formula-NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

### Methods

Disclosed herein are methods for forming a cyclopropyl or cyclopropenyl ring in a compound by, for example, contacting an unsaturated hydrocarbon and an active methylene compound with an oxidant and a catalytically effective amount of a photocatalyst; and irradiating the photocatalyst with a light source to form a cyclopropyl or cyclopropenyl-containing product.

As used herein, the term “unsaturated hydrocarbon” refers to a substituted or unsubstituted hydrocarbon containing an unsaturated bond. The unsaturated bond can be a double bond or a triple bond. In some examples, the unsaturated hydrocarbon contains a single unsaturated bond. In other examples, the number of unsaturated bonds can be two or more (e.g., two or more double bonds, two or more triple bonds, or one or more double bonds and one or more triple bonds), three or more (e.g., three or more double bonds, three or more triple bonds, two or more double bonds and one or more triple bonds, or one or more double bonds and two or more triple bonds), etc. Advantageously, the methods described herein can be used for both monocyclopropanation or multicyclopropanation (e.g., dicyclopropanation) of unsaturated hydrocarbons having multiple unsaturated bonds (e.g., two or more double bonds). The unsaturated hydrocarbon can include, for example, an unactivated alkene. The term “unactivated alkene” refers to an alkene without electron-withdrawing groups directly attached to a double bond.

In some examples, the unsaturated hydrocarbon comprises an alkene. It was determined that the described method for cyclopropanation proceeds efficiently with a variety of terminal and internal alkenes and tolerates numerous functional groups that are deleterious to conventional cyclopropanation pathways. A “terminal alkene” refers to an alkene wherein a double bond is between two carbon atoms at the end of the hydrocarbon chain. For example, the method can utilize terminal alkenes having acyclic and cyclic alkyl, and aryl backbones, with or without functional groups, such as ester, carbonate, carbamate, epoxide, ether, alkyne, alkyl bromide and aryl bromide, among others. In some examples, the alkene includes an alkene having active hydrogens on α-carbons, such as phosphates, malonate esters, and amide nitrogens. In some examples, the terminal alkene is an alkene defined by the formula R¹ (CH)CH₂, wherein
R¹ is hydrogen, a halogen, a substituted or unsubstituted aryl, a substituted or unsubstituted alkyl, a substituted or unsubstituted alkylaryl, a substituted or unsubstituted heteroaryl, a substituted or unsubstituted alkylheteroaryl, —CHO, —CO₂R^{a}, —N(R^{a})₂, or —OR^{a}; and
each R^{a}, when present, is independently a substituted or unsubstituted aryl, a substituted or unsubstituted alkyl, a substituted or unsubstituted alkylaryl, a substituted or unsubstituted heteroaryl, or a substituted or unsubstituted alkylheteroaryl. In some examples, R¹ is hydrogen. In some examples, R¹ is a halogen. In some examples, R¹ is a substituted or unsubstituted aryl (e.g., a substituted or unsubstituted heteroaryl). For example, R¹ can include substituted or unsubstituted phenyl, benzyl 2-naphthyl, 1-naphthyl, anthracene, phenanthrene, indene, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzodioxolyl, benzothiophene, indole, indazole, benzimidizolyl, imidazopyridinyl, pyrazolopyridinyl, and pyrazolopyrimidinyl. In some examples, R¹ is an alkylaryl. In some examples, R¹ is an alkylheteroaryl. In some examples, R¹ is a substituted or unsubstituted alkyl. For example, R¹ can include substituted or unsubstituted methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, or tetracosyl. In some examples, R¹ is —N(R^{a})₂, wherein each R^{a} is independently a substituted or unsubstituted alkyl or a substituted or unsubstituted aryl. In some examples, R¹ is —OR^{a} wherein each R^{a} is independently a substituted or unsubstituted alkyl or a substituted or unsubstituted aryl. Non-limiting examples of substituted or unsubstituted alkyl or substituted or unsubstituted aryls include any of the described above.

Remarkably, the described methods are further applicable to sterically challenging linear and cyclic disubstituted internal alkenes. In some examples, the alkene comprises an internal alkene. An “internal alkene” refers to an alkene wherein a double bond is between two carbon atoms that are not at the end of the hydrocarbon chain.

The present method can also be used to form a cyclopropenyl ring in a compound from an unsaturated hydrocarbon comprising an alkyne. Examples of alkynes are, but not limited to, acetylene, ethyne, propyne, butyne, hexyne, heptyne, or octyne, or an aromatic (e.g., heteroaromatic) compound substituted with an alkynyl group. In some examples, the alkyne is defined by the formula:

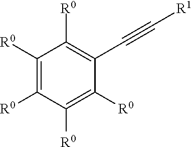

wherein R¹ is H or alkyl, and
each R⁰ is independently H, halogen, or alkyl. In some examples, R¹ is hydrogen. In some examples, R¹ is an alkyl.

For example, R¹ can be a substituted or unsubstituted C₁-C₁₀ alkyl (e.g., C₁-C₈ alkyl, C₁-C₆ alkyl, C₁-C₄ alkyl). For example, R¹ can include substituted or unsubstituted methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, or tetracosyl. In some examples, each R⁰ is the same. In some examples, at least one R⁰ is different. In some examples, R⁰ is hydrogen. In some examples, R⁰ is a halogen. In some examples, R⁰ is an alkyl (e.g., a substituted or unsubstituted C₁-C₁₀ alkyl, C₁-C₈alkyl, C₁-C₆ alkyl, or C₁-C₄ alkyl).

The term “active methylene compound” refers to a compound having two or more active methylene hydrogens which form an intermediate carbanion following hydrogen abstraction by a base. In various implementations, the active methylene compound is defined by the formula R² (CH₂) R³, wherein R² and R³ are each independently an electron withdrawing group. The term “electron withdrawing group” generally refers to a functional group that draws electrons away from a reaction center. Some non-limiting examples of electron withdrawing groups include, but are not limited to, —C(═O), —CN, —NO₂, —CX₃, —X, —COOR, —CONR₂, —COR, —COX, —SO₂R, —SO₂OR, —SO₂NHR, —SO₂NR₂, —PO₃R², —P(O)(CH₃)NHR, NO, —NR₃⁺, —CR═CR₂, and —C═CR wherein X is F, Br, Cl, or I, and R is, at each occurrence, independently selected from the group consisting of hydrogen and C₁-C₁₀ alkyl (e.g., C₁-C₈ alkyl, C₁-C₆ alkyl, C₁-C₄ alkyl). Electron withdrawing groups can also include aryl groups (e.g., phenyl) and certain heteroaryl groups (e.g., pyridine). The term “electron withdrawing groups” includes aryls or heteroaryls further substituted with electron withdrawing groups. In some examples, R² and R³ are each independently an electron withdrawing group selected from the group consisting of a halogen (e.g., F, Cl, Br, or I), —C(O)R^{b}, —COOR^{b}, —C(O)NR^{b}R^{c}, —CN, —SO₃R^{b}, —SO₂R^{b}, —SO₂NR^{b}R^{c}, —OP(O)OR^{b}OR^{c}, —R^{b}NCO, and —NO₂, wherein R^{b} and R^{c}, when present, are independently H or a substituted or unsubstituted C₁-C₁₀ alkyl (e.g., C₁-C₈alkyl, C₁-C₆ alkyl, C₁-C₄ alkyl). For example, the active methylene compound can be defined by the formula R^{b}OOC(CH₂) COOR, wherein R^{b} and R^{c} are independently H or a substituted or unsubstituted C₁-C₁₀ alkyl (e.g., C₁-C₈ alkyl, C₁-C₆ alkyl, C₁-C₄ alkyl). In some examples, one or both of R² and R³ are a halogen (e.g., F, Cl, Br, or I). In some examples, one or both of R² and R³ are —C(O)R^{b}, wherein R^{b} and R^{e} are independently H or a substituted or unsubstituted C₁-C₁₀ alkyl (e.g., C₁-C₈alkyl, C₁-C₆ alkyl, C₁-C₄ alkyl). In some examples, one or both of R² and R³ are —COOR^{b}, wherein R^{b} and R^{e} are independently H or a substituted or unsubstituted C₁-C₁₀ alkyl (e.g., C₁-C₈alkyl, C₁-C₆ alkyl, C₁-C₄ alkyl). In some examples, one or both of R² and R³ are —C(O)NR^{b}R^{c}, wherein R^{b} and R^{c} are independently H or a substituted or unsubstituted C₁-C₁₀ alkyl (e.g., C₁-C₈alkyl, C₁-C₆ alkyl, C₁-C₄ alkyl). In some examples, one or both of R² and R³ are —CN. In some examples, one or both of R² and R³ are —SO₃R^{b}, wherein R^{b} is H or a substituted or unsubstituted C₁-C₁₀ alkyl (e.g., C₁-C₈alkyl, C₁-C₆ alkyl, C₁-C₄ alkyl). In some examples, one or both of R² and R³ are —SO₂R^{b}, wherein Rbis H or a substituted or unsubstituted C₁-C₁₀ alkyl (e.g., C₁-C₈alkyl, C₁-C₆ alkyl, C₁-C₄ alkyl). In some examples, one or both of R² and R³ are —SO₂NR^{b}R^{c}, wherein R^{b} and R^{c} are independently H or a substituted or unsubstituted C₁-C₁₀ alkyl (e.g., C₁-C₈alkyl, C₁-C₆ alkyl, C₁-C₄ alkyl). In some examples, one or both of R² and R³ are —OP(O)OR^{b}OR^{c}, wherein R^{b} and R^{c} are independently H or a substituted or unsubstituted C₁-C₁₀ alkyl (e.g., C₁-C₈alkyl, C₁-C₆ alkyl, C₁-C₄ alkyl). In some examples, one or both of R² and R³ are —R^{b}NCO, wherein R^{b} is H or a substituted or unsubstituted C₁-C₁₀ alkyl (e.g., C₁-C₈ alkyl, C₁-C₆ alkyl, C₁-C₄ alkyl). In some examples, one or both of R² and R³ are —NO₂. R² and R³ can be the same or different. R^{b} and R^{c}, when present, can be the same or different. In various implementations, the active methylene compound comprises diethylmalonate.

The present method uses one or more oxidants (e.g., two or more) to facilitate the formation of the cyclopropyl or cyclopropenyl ring in the compound. In some examples, the oxidant comprises a peroxide. In some examples, the oxidant comprises an alkyl halide, including but not limited to chlorocyclohexane, bromocyclohexane, 1-iodopropane, 2-iodopropane, iodocyclopentane, and/or iodocyclohexane. For example, the oxidant can include an alkyl iodide (e.g., a cycloalkyl iodide), such as iodocyclohexane and/or iodocyclopentane. In some examples, the oxidant comprises tert-butyl peroxide. In some examples, the oxidant comprises benzoyl peroxide. In some examples, the oxidant comprises pyridine N-oxide. In some examples, the oxidant comprises K₂S₂O₈. In some examples, the oxidant comprises tert-butyl peroxybenzoate. In some examples, the oxidant comprises tert-butyl hydroperoxide. In some examples, the oxidant comprises tert-butyl hypochlorite. In some examples, the oxidant comprises sodium perchlorate. In some examples, the oxidant comprises a first oxidant and a second oxidant. The first oxidant can comprise, for example, an alkyl halide. The second oxidant can be O₂ (e.g., air). In some examples, the first and/or second oxidant is a peroxide.

The unsaturated hydrocarbon, active methylene compound, oxidant, and photocatalyst can, in some examples, be contacted in a solvent. Exemplary solvents include dimethylformamide (DMF), dioxane, dimethylacetamide (DMA), dimethyl sulfoxide (DMSO), N-methyl-2-pyrrolidone (NMP), toluene, dichloromethane, or a combination thereof. In some examples, the solvent comprises DMF.

The present method can form compounds comprising cyclopropyl and/or a cyclopropenyl in high yields. The term “yield” as used herein, refers to the empirical yield for a given chemical reaction. Yield is a percent which represents the extent to which a reaction proceeded to produce a given product (e.g., the cyclopropyl or cyclopropenyl-containing product). Percent yield is calculated by assuming a chemical reaction and assuming that all of the chemical reagents react and become products, limited only by the limiting reagent wherein the limiting reagent is the reagent which is consumed first as the reaction proceeds. For example, the method can produce a yield of the cyclopropyl or cyclopropenyl-containing product of 30% or more, such as 40% or more, 50% or more, 60% or more, 70% or more, 80% or more, 90% or more, 95% or more, or 99% or more.

In some examples, the present method can be performed at an ambient temperature (e.g., from 10° C.-50° C., from 10° C.-40° C., from 10° C.-30° C., from 20° C.-50° C., from 30° C.-50° C., or from 30° C.-40° C.). In some examples, 50% conversion of the unsaturated hydrocarbon to the product is achieved in 16 hours or less (e.g., 12 hours or less, 10 hours or less, 8 hours or less, 6 hours or less, 5 hours or less, 4 hours or less, 3 hours or less, 2 hours or less, or 1 hours or less).

In the presently disclosed methods, the photocatalyst is irradiated with a light source to form a cyclopropyl or cyclopropenyl-containing product. In some examples, the photocatalyst comprises an organic or organometallic photocatalyst. In some examples, the photocatalyst comprises 2,4,5,6-tetra(9H-carbazol-9-yl)isophthalonitrile (4CzIPN), Eosin Y salts, [Ir(dtbbpy)(ppy)₂][BF₄], [Ir(dFCF₃ppy))₂(bpy)]PF₆, Ir(ppy)₃, or a combination thereof. Irradiation of the photocatalyst generally includes supplying the photocatalyst with electromagnetic energy (e.g., light) at a wavelength suitable for a photochemical reaction. In some examples, the photocatalyst is irradiated with visible light. The term “visible light” generally refers to light having a wavelength between 390 nanometers (nm) and 750 nm. In some examples, the visible light has a wavelength of from 390 nm to 467 nm. The light source can include any natural or artificial source of irradiation sufficient to activate the photocatalyst and produce the desired cyclopropyl and/or a cyclopropenyl products. Non-limiting types of suitable light sources include, for example, lasers, mercury (Hg) UV-lamps, incandescent lamps, fluorescent tubes, plasmas or light-emitting diodes (LEDs), sunlight, or ambient room light. In some examples, the light source is a blue LED.

Also described herein are methods for forming a cyclopropyl or cyclopropenyl ring in a compound comprising contacting an unsaturated hydrocarbon and an active methylene compound with an oxidant under conditions effective to form a cyclopropyl or cyclopropenyl ring. Conditions may include temperature, pressure, reaction time, and the like as illustrated in the supporting examples.

In some examples of the present method, the unsaturated hydrocarbon comprises an alkene. For example, the unsaturated hydrocarbon can include a terminal alkene (e.g., 3-butenylbenzene). In some examples, the terminal alkene is an alkene defined by the formula R¹ (CH)CH₂, wherein
R¹ is hydrogen, a halogen, a substituted or unsubstituted aryl, a substituted or unsubstituted alkyl, a substituted or unsubstituted alkylaryl, a substituted or unsubstituted heteroaryl, a substituted or unsubstituted alkylheteroaryl, —CHO, —CO₂R^{a}, —N(R^{a})₂, or —OR^{a}; and
each R^{a}, when present, is independently a substituted or unsubstituted aryl, a substituted or unsubstituted alkyl, a substituted or unsubstituted alkylaryl, a substituted or unsubstituted heteroaryl, or a substituted or unsubstituted alkylheteroaryl. In some examples, R¹ is hydrogen. In some examples, R¹ is a halogen. In some examples, R¹ is a substituted or unsubstituted aryl (e.g., a substituted or unsubstituted heteroaryl). For example, R¹ can include substituted or unsubstituted phenyl, 2-naphthyl, 1-naphthyl, anthracene, phenanthrene, indene, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzodioxolyl, benzothiophene, indole, indazole, benzimidizolyl, imidazopyridinyl, pyrazolopyridinyl, and pyrazolopyrimidinyl. In some examples, R¹ is a substituted or unsubstituted alkyl. For example, R¹ can include substituted or unsubstituted methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, or tetracosyl. In some examples, R¹ is —N(R^{a})₂, wherein each R^{a} is independently a substituted or unsubstituted alkyl or a substituted or unsubstituted aryl. In some examples, R¹ is —OR^{a} wherein each R^{a} is independently a substituted or unsubstituted alkyl or a substituted or unsubstituted aryl. Non-limiting examples of substituted or unsubstituted alkyl or substituted or unsubstituted aryls include any of the groups described above.

In some examples, the alkene comprises an internal alkene. As described above, internal alkenes are alkenes wherein a double bond is between two carbon atoms that are not at the terminal end of a hydrocarbon chain.

The present method can also be used to form a cyclopropenyl ring in a compound from an unsaturated hydrocarbon comprising an alkyne. Examples of alkynes are, but not limited to, acetylene, ethyne, propyne, butyne, hexyne, heptyne, or octyne, or an aromatic (e.g., heteroaromatic) compound substituted with an alkynyl group. In some examples, the alkyne is defined by the formula:

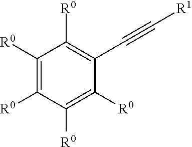

wherein R¹ is H or alkyl, and
each R⁰ is independently H, halogen, or alkyl. In some examples, R¹ is hydrogen. In some examples, R¹ is an alkyl. For example, R¹ can be a substituted or unsubstituted C₁-C₁₀ alkyl (e.g., C₁-C₈ alkyl, C₁-C₆ alkyl, C₁-C₄ alkyl). For example, R¹ can include substituted or unsubstituted methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, or tetracosyl. In some examples, each R⁰ is the same. In some examples, at least one R⁰ is different. In some examples, R⁰ is hydrogen. In some examples, R⁰ is a halogen. In some examples, R⁰ is an alkyl (e.g., a substituted or unsubstituted C₁-C₁₀ alkyl, C₁-C₈alkyl, C₁-C₆ alkyl, or C₁-C₄ alkyl).

In various implementations, the active methylene compound is defined by the formula R² (CH₂) R³, wherein R² and R³ are each independently an electron withdrawing group. In some examples, R² and R³ are each independently an electron withdrawing group selected from the group consisting of a halogen (e.g., F, Cl, Br, or I), —C(O)R^{b}, —COORD, —C(O)NR^{b}R^{c}, —CN, —SO₃R^{b}, —SO₂R^{b}, —SO₂NR^{b}R^{c}, —OP(O)OR^{b}OR^{c}, —R^{b}NCO, and —NO₂, wherein R^{b} and R^{c}, when present, are independently H or a substituted or unsubstituted C₁-C₁₀ alkyl (e.g., C₁-C₈alkyl, C₁-C₆ alkyl, C₁-C₄ alkyl). For example, the active methylene compound can be defined by the formula R^{b}OOC(CH₂) COOR^{c}, wherein R^{b} and R^{c} are independently H or a substituted or unsubstituted C₁-C₁₀ alkyl (e.g., C₁-C₈ alkyl, C₁-C₆ alkyl, C₁-C₄ alkyl). In some examples, one or both of R² and R³ are halogens (e.g., F, Cl, Br, or I). In some examples, one or both of R² and R³ are —C(O)R^{b}, wherein R^{b} and R^{c} are independently H or a substituted or unsubstituted C₁-C₁₀ alkyl (e.g., C₁-C₈alkyl, C₁-C₆ alkyl, C₁-C₄ alkyl). In some examples, one or both of R² and R³ are —COOR^{b}, wherein R^{b} and R^{c} are independently H or a substituted or unsubstituted C₁-C₁₀ alkyl (e.g., C₁-C₈alkyl, C₁-C₆ alkyl, C₁-C₄ alkyl). In some examples, one or both of R² and R³ are —C(O)NR^{b}R^{c}, wherein R^{b} and R^{c} are independently H or a substituted or unsubstituted C₁-C₁₀ alkyl (e.g., C₁-C₈alkyl, C₁-C₆ alkyl, C₁-C₄ alkyl). In some examples, one or both of R² and R³ are —CN. In some examples, one or both of R² and R³ are —SO₃R^{b}, wherein R^{b} is H or a substituted or unsubstituted C₁-C₁₀ alkyl (e.g., C₁-C₈alkyl, C₁-C₆ alkyl, C₁-C₄ alkyl). In some examples, one or both of R² and R³ are —SO₂R^{b}, wherein R^{b} is H or a substituted or unsubstituted C₁-C₁₀ alkyl (e.g., C₁-C₈alkyl, C₁-C₆ alkyl, C₁-C₄ alkyl). In some examples, one or both of R² and R³ are —SO₂NR^{b}R^{c}, wherein R^{b} and R^{c} are independently H or a substituted or unsubstituted C₁-C₁₀ alkyl (e.g., C₁-C₈alkyl, C₁-C₆ alkyl, C₁-C₄ alkyl). In some examples, one or both of R² and R³ are —OP(O)OR^{b}OR^{c}, wherein R^{b} and R^{c} are independently H or a substituted or unsubstituted C₁-C₁₀ alkyl (e.g., C₁-C₈alkyl, C₁-C₆ alkyl, C₁-C₄ alkyl). In some examples, one or both of R² and R³ are —R^{b}NCO, wherein R^{b} is H or a substituted or unsubstituted C₁-C₁₀ alkyl (e.g., C₁-C₈ alkyl, C₁-C₆ alkyl, C₁-C₄ alkyl). In some examples, one or both of R² and R³ are —NO₂. R² and R³ can be the same or different. R^{b} and R^{c}, when both present, can be the same or different. In various implementations, the active methylene compound comprises diethylmalonate.

The present method uses one or more oxidants (e.g., two or more oxidants) to facilitate the formation of the cyclopropyl or cyclopropenyl ring in the compound. In some examples, the oxidant comprises a peroxide. In some examples, the oxidant comprises an alkyl halide. For example, the oxidant can include an alkyl iodide (e.g., a cycloalkyl iodide), such as iodocyclohexane. In some examples, the oxidant comprises NaIO₄. In some examples, the oxidant comprises a first oxidant and a second oxidant. The first oxidant can comprise, for example, an alkyl halide. The second oxidant can be O₂ (e.g., air). In some examples, the second oxidant is a peroxide.

The unsaturated hydrocarbon, active methylene compound, and oxidant can, in some examples, be contacted in a solvent. Some suitable solvents include dimethylformamide (DMF), dioxane, dimethylacetamide (DMA), dimethyl sulfoxide (DMSO), N-methyl-2-pyrrolidone (NMP), toluene, dichloromethane, or a combination thereof. In some examples, the solvent comprises DMF.

The present method can form compounds comprising cyclopropyl and/or a cyclopropenyl in high yields. For example, the method can produce a yield of the cyclopropyl or cyclopropenyl-containing product of 30% or more, such as 40% or more, 50% or more, 60% or more, 70% or more, 80% or more, 90% or more, 95% or more, or 99% or more.

In some examples, the present method can be performed at an ambient temperature (e.g., from 10° C. to 50° C., from 10° C. to 40° C., from 10° C. to 30° C., from 20° C. to 50° C., from 30° C. to 50° C., or from 30° C. to 40° C.). In some examples, 50% conversion of the unsaturated hydrocarbon to the product is achieved in 16 hours or less (e.g., 12 hours or less, 10 hours or less, 8 hours or less, 6 hours or less, 5 hours or less, 4 hours or less, 3 hours or less, 2 hours or less, or 1 hours or less).

Also described herein are compounds comprising a cyclopropyl or cyclopropenyl ring formed by the above methods.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1

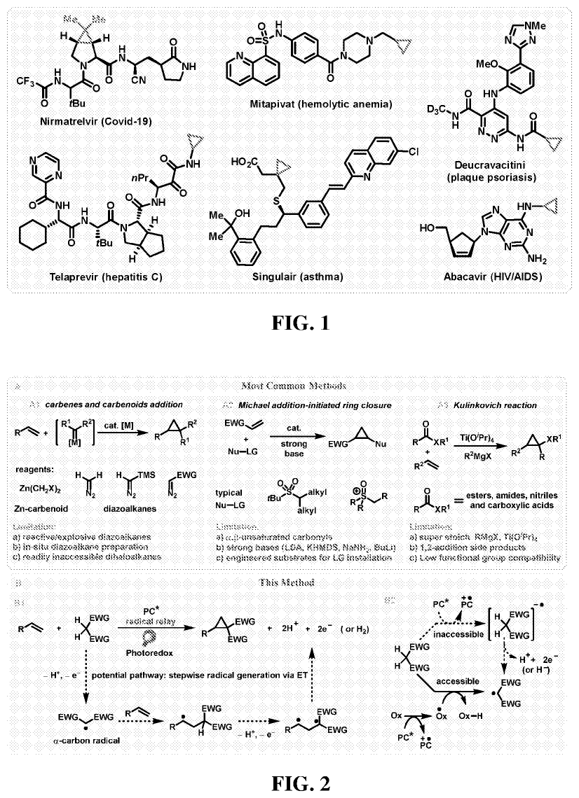

As a prominent member of strained cycloalkanes, cyclopropane is forged as a highly valuable and versatile intermediates in the synthesis of complex molecules and natural products.¹⁻³ Cyclopropyl moieties are also extensively featured as a pivotal design element in preclinical and clinical drug molecules⁴ to achieve specific therapeutic goals, and a key motif in biologically active molecules⁵ and natural products.^{6,7} For example, the recently FDA-approved drugs, Nirmatrelvir (PAXLOVID™) for Covid-19, Mitapivat (PYRUKYND®) for hemolytic anemia, Deucravacitinib (SOTYKTU™) for plaque psoriasis, and 14 out of 200 top-selling small molecule commercial drugs,⁴ including Singulair (asthma), Telaprevir (Hepatitis C) and Abacavir (HIV/AIDS) (FIG. 1), contain a cyclopropyl ring as a key element. The undertaking for cyclopropane-loaded drugs is on a continuous rise and, therefore, cyclopropanation is one of the most investigated strained ring forming reactions in organic synthesis. Yet, the carbonaceous triangle is also among the most difficult rings to create because of the ring and torsional strains, which destabilize the ring (by 17 kcal/mol) and cause it to ring-open during reactions.

Among limited methods available for intermolecular cyclopropanation (FIG. 2, panel A),⁸ the most utilized and reliable method is the carbene insertion into alkenes (FIG. 2, panel A1).^{9,10} This method is profoundly dependent on reactive carbene and metal-carbenoid intermediates [[M]=CR₂], in particular the donor-acceptor Rh-carbenoids,⁹⁻¹¹ derived from highly energetic and explosive diazoalkanes, which require stringent precautions for handling. The pre-carbenoid diazoalkanes, such as diazomethane (CH₂N₂), TMSCHN₂, PhCHN₂, and α-diazocarbonyl compounds and their analogs, are generally subjected to decomposition upon transition-metal [M] catalysts,^{8,12,13} and more recently under photoredox^{14,15} and engineered enzymatic¹⁶⁻¹⁹ conditions to generate [M]=CR₂ intermediates in situ. Likewise, the classic Simmons-Smith and related reactions²⁰ demonstrate impressive ability to translate alkenes into cyclopropanes. Yet again, these reactions require a large excess of both unstable, and frequently difficult-to-access, 1,1-dihaloalkanes and highly reactive Et₂Zn or Zn/Cu to generate an adequate quantity of zinc carbenoids for satisfactory reactivity.

Alternatively, alkenes are also often intermolecularly cyclopropanated by means of non-carbenoid chemistry, such as the Johnson-Corey-Chaykovsky reaction,²¹ via a Michael addition-initiated ring closure (FIG. 2, panel A2).^{22,23} This process usually requires a careful molecular engineering to install a leaving group at a precise reaction site either on Michael acceptors or Michael donors and a strong base, such as LDA, KHMDS, NaNH₂ or BuLi, to enolize the Michael donors for sufficient nucleophilicity. This method is also characteristically limited to α,β-unsaturated carbonyls, and unactivated alkenes are beyond its scope. Additionally, the Kulinkovick reaction (FIG. 2, panel A3)²⁴⁻²⁶ converts carbonyl compounds and terminal alkenes into cyclopropyl alcohols and amines. Regrettably, the method suffers from functional group incompatibility and 1,2-addition reactions because of the necessity to use greater than stoichiometric amounts of titanium salts and alkyl Grignard reagents to generate sufficient quantity of titanacyclopropanes in situ as cyclopropanating reagents.

Recently, emerging catalytic methods have sought to simplify cyclopropanation of alkenes with simple organic precursors (FIG. 2, panel B).²⁷ These processes also pivot on the generation and exploitation of [M]=CR₂ intermediates²⁸⁻³¹ in situ and provide a cyclopropanation method that is more synthetically amenable and practical than diazoalkane-dependent methods. However, these methods are generally applicable to styrene and 1,3-diene derivatives and often require specialized reagents and a few-step synthetic manipulations to arrive at reactive [M]=CR₂ intermediates in situ. Evidently, the catalytic intermolecular construction of cyclopropyl rings on unactivated alkenes conveniently with simple, readily available, bench-stable and non-explosive chemicals that is also operationally straightforward and broadly applicable is an ongoing challenge in organic synthesis.³²⁻³⁵

Disclosed in this example is a simple photoredox approach for the intermolecular cyclopropanation of unactivated alkenes directly with active methylene compounds through an O₂/alkyl halide electron relay pair system (FIG. 2, panel B). This method offers a convenient protocol with bench-stable chemicals under neutral reaction conditions in air/O₂ without requiring special safety and sensitivity precautions (such as stringent N₂ environment and glovebox). In addition, since hundreds of active methylene compounds with varied functional substituents are commercially available and an almost unlimited number of their analogs can be readily synthesized and stored for prolonged periods for use on demand, the current reaction provides a simplistic, practical, and broad-scope method for the intermolecular cyclopropanation of unactivated alkenes.

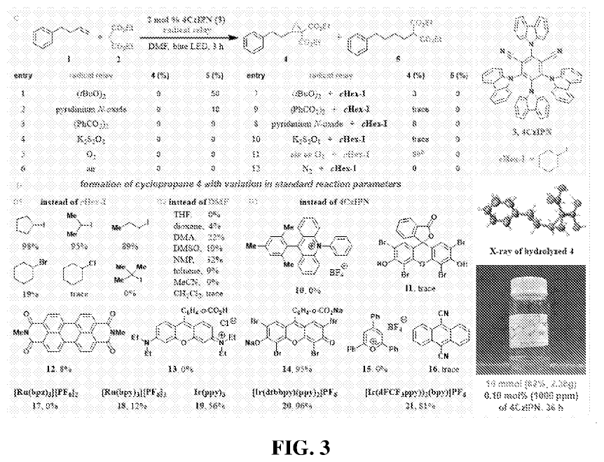

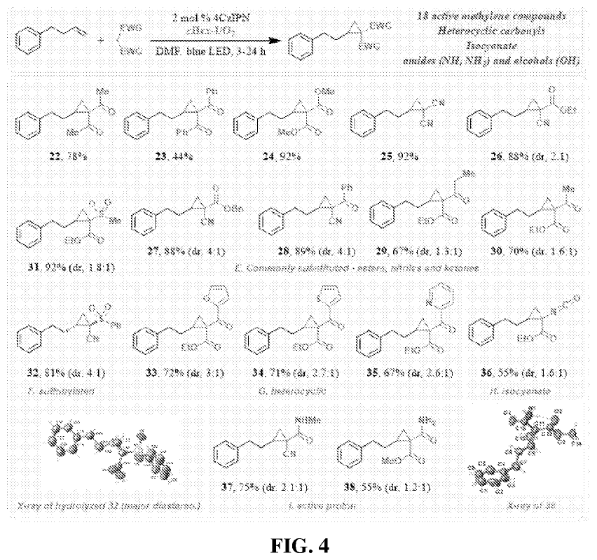

The direct cyclopropanation of alkenes with active methylene compounds includes the removal of two protons and two electrons (or H₂) through redox chemistry (FIG. 2, panel B1). Without wishing to be bound by theory, it was believed that such a process could be mediated by an excited state photocatalyst (PC*) via stepwise electron transfer (ET) and the generation of α-carbon-centered radicals (FIG. 2, panel B2). Since the direct ET reduction of an active methylene group to remove a hydride might not be amenable from an acidic α-carbon center from a thermodynamic standpoint, the study opted for the intermediacy of photoexcited oxidants to generate high energy radicals to function as radical relay for α-H abstraction (FIG. 2, panel B2). Accordingly, the experiment initially examined a range of common peroxide oxidants with 4CzIPN as a photocatalyst (PC) under a blue LED light to generate intermediary oxygen/carbon-centered radicals to subsequently abstract α-H atom from an active methylene set (FIG. 3, panel C) (entries 1-6). When 4-phenylbutene and diethyl malonate were taken as reactants, di-tert-butyl peroxide and pyridiunium N-oxide facilitated hydroalkylation with no cyclopropanation products observed (entries 1-2). Use of oxygen, a photosensitizable oxidant to generate a superoxide anion,³⁶ also didn't produce any cyclopropanated product (entry 5). However, the experiment discovered that a combination of peroxide oxidants and alkyl iodides, such as cyclohexyl iodide (cHex-I), a radical relay pair known to generate highly reactive carbon-centered radicals,³⁷ began to furnish the cyclopropanated product 4 in observable amounts (entries 7-10). Remarkably, when O₂ was combined with cHex-I, the photocatalytic reaction furnished the cyclopropanated product 4 in quantitative yield in 3 h (entry 11). The formation of the cyclopropyl ring was unambiguously established by a single crystal X-ray analysis of hydrolyzed product 4 as dicarboxylic acid. The O₂/alkyl iodide combination was recently shown to generate carbon-centered radicals under electrocatalysis,³⁸ which was effective in abstracting an α-H from methylene compounds. Control experiments under N₂ and in the absence of alkyl iodide furnished no product (entries 5, 12), indicating that photoexcited dioxygen functioned as an electron transporter from the PC* to reduce alkyl iodide in a radical relay process and generate alkyl radicals to abstract α-H from the active methylene group.

Further studies indicated that the reaction could be performed simply in air, albeit requiring a longer reaction time (16 h) (entry 11). Reactions can be conducted with 0.10 mol % (1000 ppm) of the photocatalyst in a small scale (0.10 mmol), which furnishes the product in quantitative yield in 3 h. Large scale (10 mmol) reactions can also be performed with 1000 ppm photocatalyst under the current reaction conditions albeit requiring a much longer reaction time (36 h) and with a slight decrease in the yield (82%, 2.38 g). Other alkyl iodides, except tert-alkyl iodide that could readily undergo elimination, could be used instead of cyclohexyl iodide (FIG. 3, panel D1). However, alkyl bromides and chlorides were not effective under the current experimental conditions. The reaction proceeded most effectively in DMF, and other polar and mid-polar solvents were either less effective or generated the cyclopropyl product 4 in lower yields (FIG. 3, panel D2). Next, the study examined different organic and organometallic PC's with a range of redox ability for their efficacy. Among several common PC's examined (FIG. 3, panel D3), Eosin Y sodium salt (11), [Ir(dtbbpy) (ppy)₂][BF₄] (20) and [Ir(dFCF₃ppy))₂ (bpy)]PF₆ (21) furnished the product 4 in high yields while Ir(ppy)₃ (19) was moderately effective. Reactions were conducted in 0.10 mmol scale, unless stated otherwise, in 0.50 mL DMF at 440 nm blue LED (36 Watt Kessil lamp at 100% intensity) and ambient temperature (~35° C. controlled by fans) in 1 dram capped glass vials. Yields were determined by GC with trimethoxybenzene as a standard.^{b}3 h in O₂ and 16 h in air.

With optimized reaction conditions, the experiment explored the scope of the cyclopropanation reaction with regard to the active methylene compounds (FIG. 4). The reaction demonstrated a remarkably broad scope on the applicability of the active methylene compounds, 18 in total categorized in 5 different clusters from the standpoint of synthetic tolerability, and generated a suite of 1,1-dicarbonylcyclopropanes. These active methylene compounds are commercially available and contain a combination of diverse synthetically valuable functional groups on the two termini of the active methylene set. Initially, the experiment examined the reactivity of the most common active methylene compounds (E) bearing esters, ketones and nitriles with the same or different functional integrity on the two termini of the methylene sets (22-30). The reactions with methylene sets bearing different functionalities, such as cyanoesters, ketoesters and ketonitriles (26-30), proceeded with moderate to good diastereoselectivity to generate trisubstituted cyclopropanes (dr, up to 4:1). The reaction also displayed high efficacy with sulfonylated methylene sets and afforded the trisubstituted sulfonylcyclopropane products (31-32) in high yields and good diastereoselectivity (F). An X-ray analysis of the hydrolyzed product of the major disastereomer of sulfonyl cyclopropane 32 revealed that the larger groups, the sulfonyl and the alkyl, were disposed in an anti-fashion along the cyclopropyl plane confirming steric control for diastereoselectivity. The reaction parameters were also amenable for the introduction of carbonylated heterocycles on cyclopropyl cores as demonstrated by the formation of products from active methylene sets bearing furanyl, thiophenyl and pyridyl rings along with esters (33-35) with moderate to good diastereoselectivity (G). Interestingly, the present method also tolerates with competence some of the most sensitive functional groups. For example, isocyanate (H) can be readily introduced into cyclopropane rings (36), an important building block in pharmaceutical and medicinal chemistry to introduce cyclopropyl amides (anticancer drug Lenvatinib³⁹) and cyclopropyl urea anologs^{40,41} (NPR-A agonist⁴²), for which no direct method exists currently, and is synthesized in three steps commencing with the cyclopropanation of acrylic esters with diazomethane. In addition, β-carbonyl 1° and 2° amides containing active hydrogens are also compatible for the cyclopropanation of alkenes (I), which generate cyclopropyl carboxamides (37-38) in excellent yields with moderate diastereoselectivity. The products of the current process, such as 1,1-dicarbonyl- and 1-carbonyl-1-sulfonylcyclopropanes, are known to function as inhibitors against a range of biological targets including BACE inhibitors for Alzheimer's disease 43,44,45 and, therefore, represent significant design elements in drug discovery. Reactions were conducted in 1.0 mmol scale, unless stated otherwise, in 5.0 mL DMF at 440 nm blue LED (36 Watt Kessil lamp at 100% intensity) and ambient temperature (~35-40° C. controlled by fans) under O₂ in 6 dram capped glass vials. Reported yields are for isolated products. dr was determined by 1H NMR.

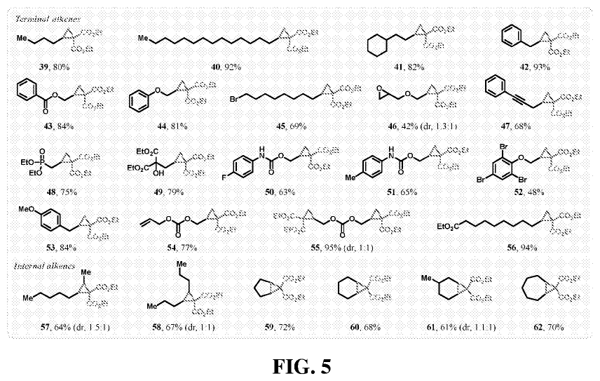

Next, the example examined the scope of the cyclopropanation reaction on alkenes taking diethyl malonate as a representative active methylene compound (FIG. 5). The reaction proceeds efficiently with terminal alkenes containing acyclic and cyclic alkyl, and aryl backbones, and tolerates various functional groups, such as ester, carbonate, carbamate, epoxide, ether, alkyne, alkyl bromide and aryl bromide (39-56). Moreover, alkenes bearing active hydrogens on α-carbons, such as phosphates (48) and malonate esters (49), and on amide nitrogen, like secondary carbamates (50-51), are also excellent substrates for cyclopropanation. The hydroxy group in the product 49 arises from further hydroxylation of an active methine hydrogen under the oxidizing condition. The reaction can be controlled for monocyclopropanation on substrates containing two alkenes (54) and is also applicable for dicyclopropanation on both alkenes (55) further highlighting the synthetic utility for selective cyclopropanation. This method also works well for the cyclopropanation of more sterically challenging linear and cyclic disubstituted internal alkenes, which generated tetrasubstituted cyclopropyl products (57-62). Reactions were conducted in 1.0 mmol scale, unless stated otherwise, in 5.0 mL DMF at 440 nm blue LED (36 Watt Kessil lamp at 100% intensity) and ambient temperature (~35-40° C. controlled by fans) under O₂ in 6 dram capped glass vials. Reported yields are for isolated products. dr was determined by ¹H NMR.

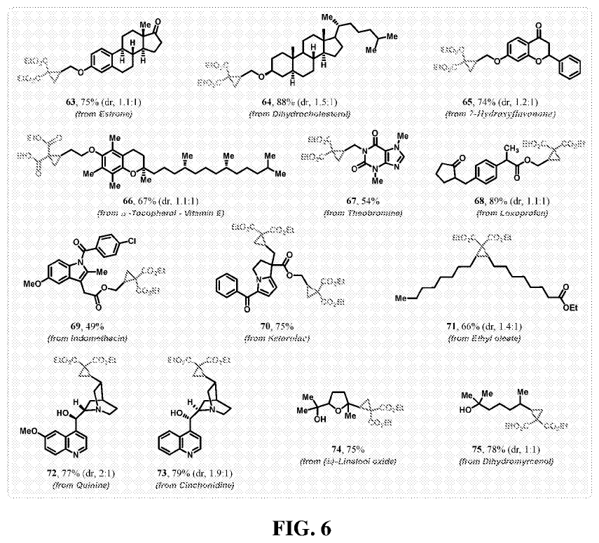

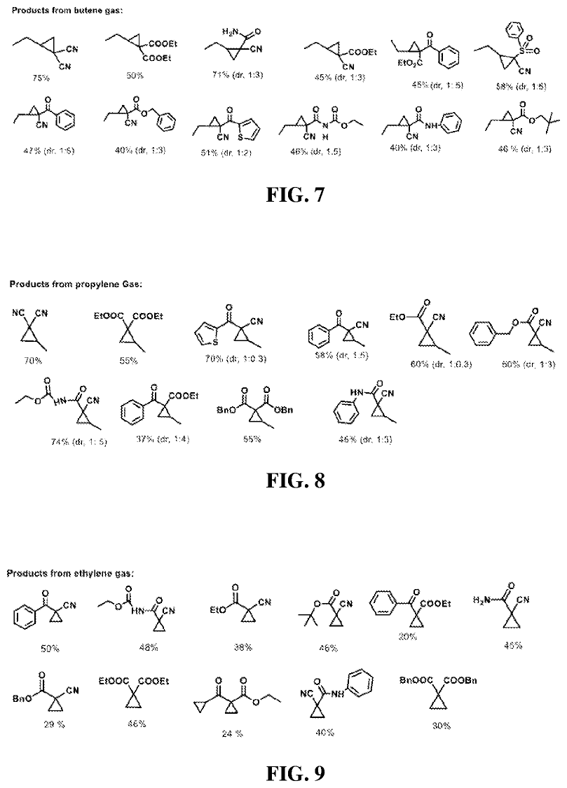

The experiment also examined the scope of the reaction on more complex structures, pharmaceuticals and natural products bearing alkenes (FIG. 6). The alkenes in O-allylated steroids estrone (63) and dihydrocholesterol (64), and O-allylated 7-hydroxyflavonone (65) and α-tocopherol (vitamin E) (66) generated cyclopropanated products in good to excellent yields. N-allylated alkaloid heterocycle theobromine (67), and O-allylated nonsteroidal anti-inflammatory drugs (NSAIDs) loxoprofen (68) and indomethacin (69) were similarly cyclopropanated with diethyl malonate. The C- and O-diallylated NSAID ketorolac (70) was efficiently converted to a product with two trisubstituted cyclopropyl rings. The naturally occurring internal alkene in the fatty acid ester ethyl oleate (71) also furnished tetrasubstituted cyclopropyl product in good yield. Additional innate alkenes in aliphatic and heterocyclic natural products bearing a free hydroxy group, such as alkaloids quinine (72) and cinchonidine (73), and terpenes linalool oxide (74) and dihydromyrcenol (75), underwent efficient cyclopropanation without interference from the unprotected alcohols present in the molecular backbones. Reactions were conducted in 1.0 mmol scale, unless stated otherwise, in 5.0 mL DMF at 440 nm blue LED (36 Watt Kessil lamp at 100% intensity) and ambient temperature (~35-40° C. controlled by fans) under O₂ in 6 dram capped glass vials. Reported yields are for isolated products. dr was determined by ¹H NMR.

In summary, the current example shows the development of a simple visible light photoredox protocol for intermolecular cyclopropanation of unactivated alkenes with active methylene compounds. The cyclopropanation is enabled by a photoredox catalyst excited under blue LED in ambient conditions with air/O₂, barring requirements of stringent safety and precautionary protocols for this class of reaction. Without wishing to be bound by theory, the reaction proceeds via a cooperative electron relay process in which a combination of an alkyl iodide and photosensitized dioxygen functions as an electron transport mechanism to transit electrons from PC* to active methylene sets for the generation of α-carbon radical, upon α-H abstraction, that appends to an alkene. The reaction demonstrates a remarkably broad scope with the participation of 18 different active methylene compounds bearing a diverse set of functionality, works for terminal and internal alkenes, and tolerates a great variety of synthetically important, and often challenging, functional groups. A broader synthetic applicability was also demonstrated by the cyclopropanation of alkenes in complex molecules including pharmaceuticals and natural products.

### Example 2

When X=H, Ar, Cycloalkane, Cl, Br, NR₂³, OR³ (R³=alkyl, aryl), C═O, CO₂R′; R=H; R¹, R²=electron-withdrawing groups such as CO₂R′, CN, COR′, isocyanates, phosphates, sulfonates; [O]=oxidants such as air, oxygen (O₂), MnO₂, NaIO₄

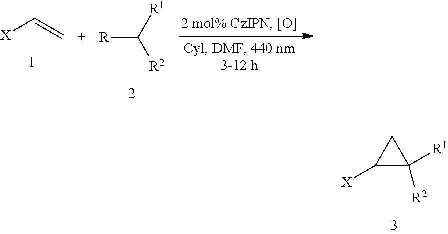

### Air or Oxygen as an Oxidant

Procedure A: An oven-dried 4 mL vial was charged with a magnetic stir bar and photocatalyst (4CzIPN, 2 mol %, 1.8 mg). Corresponding alkene 1 (0.1 mmol, 1 eq), active methylene compound 2 (0.2 mmol, 2 eq), iodocyclohexane (0.1 mmol, 1 eq), and anhydrous DMF (0.5 mL) was added to the vial in both in air and under 02. The resulting mixture was then irradiated with KESSIL blue LEDs (36 W operating at 100% intensity) (390 nm to 467 nm) under air or O₂ atmosphere. After 3-12 hours, the reaction mixture was poured into water and extracted with EtOAc (3×), combined organic layers were washed with water (5×), dried over anhydrous Na₂SO₄, and concentrated in vacuo. The yields of the product 3 was found to be 58%-99%.

### MnO2 as an Oxidant

Following the above procedure A, manganese dioxide was used as an oxidant (3 eq) instead of molecular oxygen. The reaction performed both in air and under O₂ atmosphere furnished the desired product with identical yields (>99%, GC).

### NaIO₄ as an Oxidant

Following the procedure A, sodium periodate was used as an oxidant (3 eq) instead of molecular oxygen. The reaction performed both in air and under O₂ atmosphere furnished the desired product with identical yields (>99% yield).

NaIO₄ or MnO₂ as an Oxidant without Using Iodocyclohexane

Following the procedure A, sodium periodate or manganese dioxide was used as an oxidant (3 eq) without using iodocyclohexane. The reaction was performed under N₂ atmosphere to furnish the desired product (>95% yield).

NaIO₄ or MnO₂ as an Oxidant without Using Iodocyclohexane and PC

Following the procedure A, sodium periodate or manganese dioxide was used as an oxidant (3 eq.) without using both iodocyclohexane and 4CzIPN. The reaction was performed under N₂ atmosphere to furnish the desired product (>90% yield).

### Example 3

When X=H, Ar, Cycloalkane, Cl, Br, NR₂³, OR³ (R³=alkyl, aryl), C═O, CO₂R′; R=Br; R¹, R²=electron-withdrawing groups such as CO₂R′, CN, COR′, isocyanates, phosphates, sulfonates.

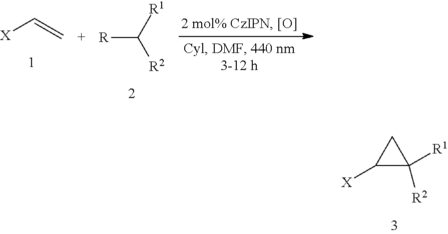

An oven-dried 4 mL vial was charged with a magnetic stir bar and photocatalyst (4CzIPN, 2 mol %, 1.8 mg). Corresponding alkene 1 (0.1 mmol, 1 eq), bromo methylene compound 2 (0.2 mmol, 2 eq), and anhydrous DMF (0.5 mL) was added to the vial in both in air and under O₂. The resulting mixture was then irradiated with KESSIL blue LEDs (36 W operating at 100% intensity) (390 nm to 467 nm). After 3 hours, the reaction mixture was poured into water and extracted with EtOAc (3×), combined organic layers were washed with water (5×), dried over anhydrous Na₂SO₄, and concentrated in vacuo. The yield of the desired product was found to be 64%.

When X=H, alkyl, Ar, Cl, Br, NR₂³, OR³ (R³=alkyl, aryl), R=Br, R¹=alkyl, aryl, H and R²=aryl are substituents in the above Scheme.

An oven-dried 4 mL vial was charged with a magnetic stir bar and photocatalyst (4CzIPN, 2 mol %, 1.8 mg). Corresponding alkene 4 (0.1 mmol, 1 eq), benzyl bromide 5 (0.2 mmol, 2 eq), and anhydrous DMF (0.5 mL) was added to the vial under N₂ atmosphere. The resulting mixture was then irradiated with KESSIL blue LEDs (36 W operating at 100% intensity) (390 nm to 467 nm). Reaction was either performed in air or in presence of O₂ (balloon). After 3 hours, the reaction mixture was poured into water and extracted with EtOAc (3×), combined organic layers were washed with water (5×), dried over anhydrous Na₂SO₄, and concentrated in vacuo. The crude was purified by silica gel column chromatography. The yield of the desired product was found to be 52-80%.

### Example 4

When R³=H, alkyl; R=H; R¹, R²=electron-withdrawing groups such as CO₂R′, CN, COR′, isocyanates, phosphates, sulfonates; [O]=oxidants such as air, oxygen (O₂), MnO₂, NaIO₄

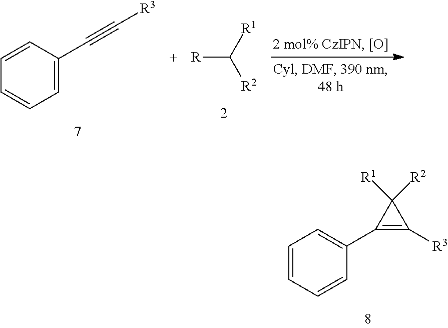

An oven-dried 4 mL vial was charged with a magnetic stir bar and photocatalyst (4CzIPN, 2 mol %, 1.8 mg). Corresponding alkyne 7 (0.5 mmol, 1 eq), active methylene compound 2 (1.0 mmol, 2 eq), iodocyclohexane (1.0 mmol, 1 eq), and anhydrous DMF (2.5 mL) was added to the vial in both in air. The resulting mixture was then irradiated with KESSIL blue LEDs (36 W operating at 100% intensity) (390 nm to 467 nm) under 02 atmosphere. After 48 hours, the reaction mixture was poured into water and extracted with EtOAc (3×), combined organic layers were washed with water (5×), dried over anhydrous Na₂SO₄, and concentrated in vacuo. The crude was purified by silica gel column chromatography to obtain desired product 8 (45%-62% yield).

### Example 5

An oven dried Schlenk tube was charged with a magnetic stir bar, methylene compound (0.5 mmol, 1 eq), NaIO₄ (3 eq), and NMP (2.5 mL) under N₂ atmosphere. Butene gas (excess) was passed into the tube and the reaction mixture was irradiated with KESSIL blue LEDs (390-440 nm, 36 W operating at 100% intensity). After 3-48 hours, reaction solution was poured into water and extracted with EtOAc (3×), washed with water (5×), concentrated, and purified by silica gel flash chromatography to obtain the desired product (37%-74% isolated yield).

### Example 6

An oven dried Schlenk tube was charged with a magnetic stir bar, methylene compound (0.5 mmol, 1 eq), NaIO₄ (3 eq), and NMP (2.5 mL) under N₂ atmosphere. Propylene gas (excess) was passed into the tube and the reaction mixture was irradiated with KESSIL blue LEDs (390-440 nm, 36 W operating at 100% intensity). After 3-48 hours, reaction solution was poured into water and extracted with EtOAc (3×), washed with water (5×), concentrated, and purified by silica gel flash chromatography to obtain the desired product (37%-74% isolated yield).

### Example 7

An oven dried Schlenk tube was charged with a magnetic stir bar, methylene compound (0.5 mmol, 1 eq), NaIO₄ (3 eq), and NMP (2.5 mL) under N₂ atmosphere. Ethylene gas (excess) was passed into the tube and the reaction mixture was irradiated with KESSIL blue LEDs (390-440 nm, 36 W operating at 100% intensity). After 3-48 hours, reaction solution was poured into water and extracted with EtOAc (3×), washed with water (5×), concentrated, and purified by silica gel flash chromatography to obtain the desired product (37%-74% isolated yield).

### Example 8

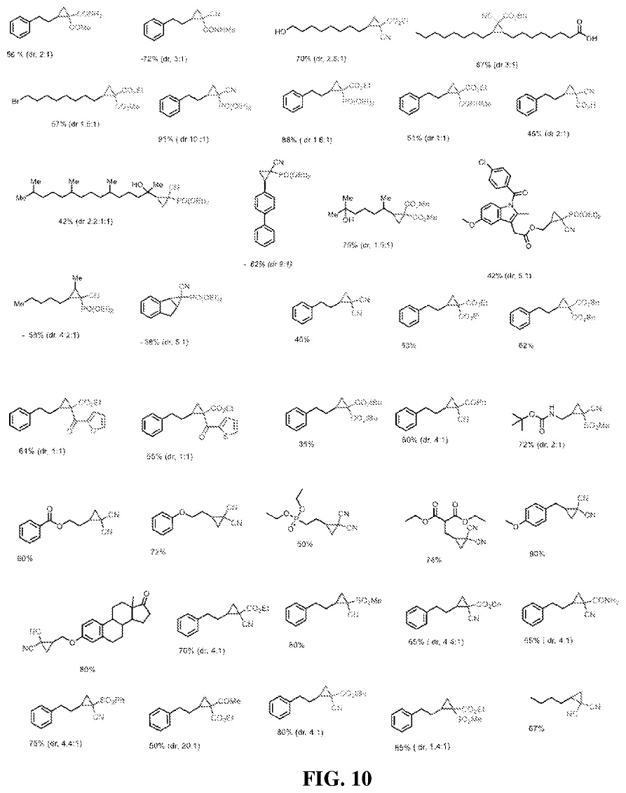

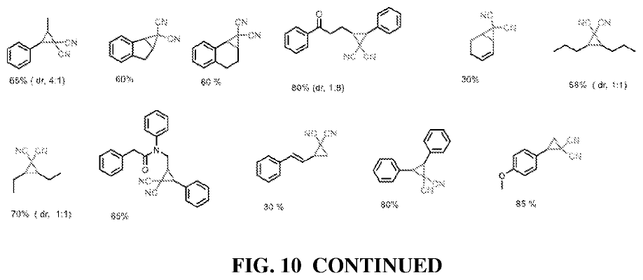

An oven-dried 4 mL vial was charged with a magnetic stir bar and photocatalyst (4CzIPN, 2 mol %, 1.8 mg). An unactivated alkene (0.1 mmol, 1 eq), an active methylene compound (0.2 mmol, 2 eq), iodocyclohexane (0.1 mmol, 1 eq), and anhydrous DMF (0.5 mL) were added to the vial in both in air and under 02. The resulting mixture was then irradiated with KESSIL blue LEDs (36 W operating at 100% intensity) (390 nm to 467 nm) with NaIO₄ as an oxidant (3 eq.). After 3-12 hours, the reaction mixture was poured into water and extracted with EtOAc (3×), combined organic layers were washed with water (5×), dried over anhydrous Na₂SO₄, and concentrated in vacuo. The resulting cyclopropanated products along with their corresponding yields and diastereomer ratios are shown in FIG. 10.

### Example 9: Mechanistic Studies

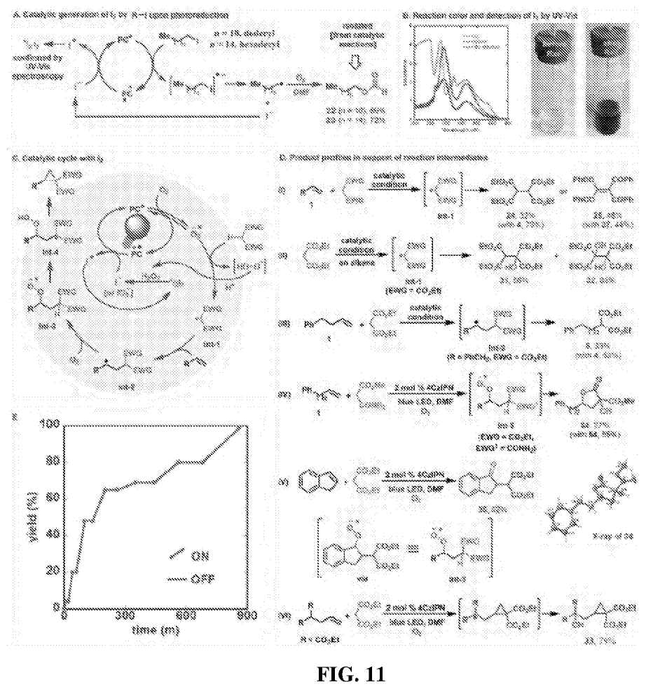

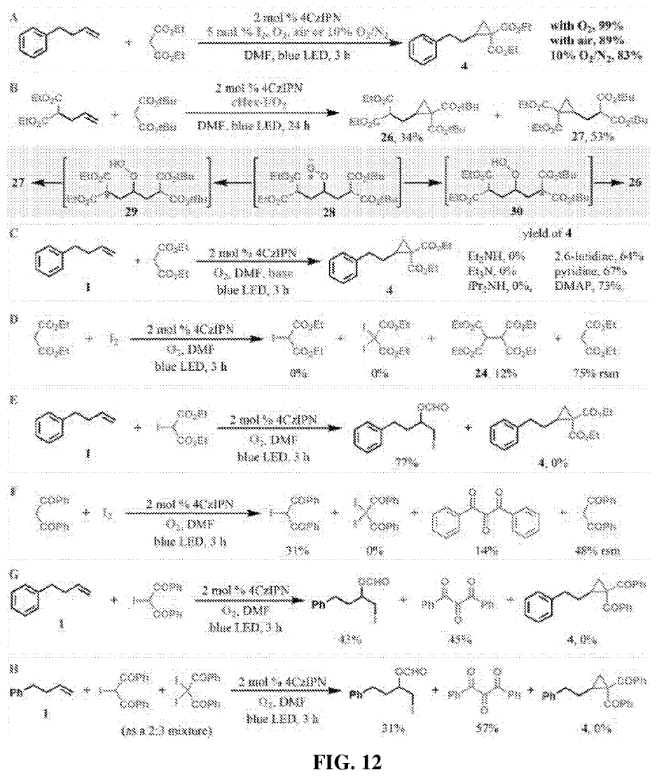

Prompted by the observed roles of alkyl iodides and O₂ for catalytic turnover, the study ventured to further study the reaction mechanism. Without wishing to be bound by theory, analysis of solutions of PC, alkyl iodide and 4-phenylbutene (1) in DMF in different combinations by UV-Vis and fluorescence spectroscopies indicated that there was no interaction of alkyl iodide with either the PC or alkene 1. Independent reactions conducted with dodecyl and hexadecyl iodides under the standard catalytic conditions confirmed the formation alkyl formates (22, 65%; 23, 72%) generated from the reaction of alkyl radicals with O₂ in DMF along with the cyclopropyl product 4 and unreacted alkyl iodides in 78-85% and 23-32%, respectively (FIG. 11, panel A). Since reaction solutions usually turned reddish brown during reaction, the experiment further analyzed reaction mixtures by UV-Vis spectroscopy and confirmed the generation of I₂ (FIG. 11, panel B). These experiments suggested that alkyl radicals are not involved in the reduction of PC radical cations for catalytic turnover. Rather iodide (I⁻) or iodate (IO₃⁻), generated from the hydrolysis or the oxidation of I₂, furnish the reactionary electrons to reduce the intermediary PC radical cations. In order to further confirm the role of I₂, the study conducted the standard catalytic reaction by replacing cHex-I with 5 mol % 12. Surprisingly, the reaction generated the cyclopropyl product 4 in 99%, 89% and 83% yields under O₂, air and 10% O₂ in N₂ respectively in 3 h (FIG. 12, panel A).

A catalytic cycle (FIG. 11, panel C) was then proposed for the cyclopropanation reaction based on the above findings and a series of additional experiments and characterization of 13 different products supporting the existence of various catalytic intermediates as outlined in FIG. 11, panel D. Without wishing to be bound by theory, it is believed that dioxygen is first reduced by photoexcited PC* to superoxide ion (O₂*), which abstracts α-H from active methylene compounds to generate α-C-radicals. The study has confirmed the formation of these a-C radicals by the isolation of the saturated dimer 24 and alkene dimer 25 as side products from catalytic reactions. Since both the cyclopropyl product 4 and the alkene dimer 25 could also arise from carbene intermediates, an intramolecular competition experiment was designed in which diethyl α-allyl malonate bearing a methine (α-C—H) group was exposed under the standard conditions to di-tert-butyl malonate containing a methylene (α-CH₂) group (FIG. 12, panel B). The reaction yielded a 2:3 mixture of the expected cyclopropyl product 26 along with another cyclopropyl product 27 derived from the innate methine group, indicating that carbene intermediates are not generated in the reaction. In the absence of alkene, the α-C radicals undergo radical dimerization followed by further oxidation to generated mono- and dihydroxylated products (31, 32). Additionally, when a reaction of diethyl α-allyl malonate with diethyl malonate was allowed to continue, α-hydroxylated cyclopropyl product 33 was generated, suggesting that the superoxide ion (O₂*) or the radical anions are capable of abstracting α-H from active methylene compounds (such as in Int-1, Int-3 and Int-4).

The α-C-radicals then most likely undergo addition to alkenes to form secondary C-radicals, which subsequently react with dioxygen to generate peroxy radical anions. The radical addition steps for the formation of Int-2 and Int-3 are supported by the isolation of hydroalkylation product 5 from the standard reaction under reduced O₂ concentration (air), and a hydroxylactone 34 via deaminative oxidation when malonamide was used. In addition, a reaction of indene with diethyl malonate generated an oxo-product 35 further confirming the existence of Int-3 in solution. The radical anions (Int-3) then abstract the intramolecular α-H to generate α-C radicals, which then subsequently undergo radical 1,3-substitution with the peroxide to create the cyclopropyl ring. An on-off experiment in which the product was formed only when the reaction was exposed to the blue LED (FIG. 11, panel E) rendered an efficient radical chain propagation mechanism unlikely.

Since the reaction protocol involves catalytic I₂, the study also interrogated other mechanistic possibilities by which photoredox catalysis operates for cyclopropanation with halogenated carbon sources. Photoredox alkene cyclopropanation reactions with halogenated carbon sources have been previously proposed to proceed by atom transfer radical addition (ATRA). In particular, a photoredox benzothiazinoquinoxaline-catalyzed cyclopropanation of alkenes with α-bromo-β-ketoesters and α-bromomalonates via a radical ATRA followed by a base-promoted intramolecular 1,3-S_{N}2 process was recently reported. Others have reported a photocatalytic intramolecular cyclopropanation of α-bromodicarbonyls with a pendant alkene, which proceeded by an ATRA/1,3-S_{N}2 sequence. Others have observed a radical-polar crossover mechanism in photoredox alkene cyclopropanation with an iodomethylsilicate. In addition, others have reported cyclopropanation of alkenylarenes with an α,α-diiodocarboxylate, a reagent which generates α-iodo-α-C radicals with potential for cyclopropane ring closure by carbeniod intermediates and an 1,3-S_{N}2 displacement. Cyclopropanation by 1,3-S_{N}2 substitution typically requires a base for the generation of a carbon nucleophile by deprotonation. However, the presently disclosed cyclopropanation reactions can proceed without the addition of bases. For example, 2,6-lutidine, a base critical for cyclopropanation by 1,3-S_{N}2 substitution, actually reduced the yield of the cyclopropanated product 4 by 35% (FIG. 12, panel C). 4-Dimethylaminopyridine (DMAP) and pyridine lowered the reaction yield, while Et₂NH, Et₃N and iPr₂NH completely suppressed the cyclopropanation reaction. Analysis of the catalytic reaction mixtures by GC, GCMS and LCMS indicated no formation of α-iodomalonate or α,α-diiodomalonate as intermediates. A control experiment attempting reaction of 0.5 equiv. I₂ with diethyl malonate under our catalytic conditions in the absence of an alkene also showed no formation of α-iodo- and α,α-diiodomalonates, and unreacted diethyl malonate was recovered (75%) (FIG. 12, panel D). The use of α-iodomalonate as a reactant also failed to generate cyclopropyl product 4 under the standard conditions. Rather, the reaction formed a 1,2-iodoformylated product (FIG. 12, panel E). Surprisingly, a similar control experiment with a 1,3-diketone (dibenzoylmethane) in the presence of I₂ without an alkene generated α-iododiketone in 31% yield along with a 1,2,3-trione (14%) and unreacted 1,3-diketone (48%) (FIG. 12, panel F). However, the reactions of α-iododiketone alone or a mixture of α-iododiketone and α,α-diiododiketone (2:3 ratio) under the standard reaction conditions generated not the cyclopropanated product 4 but rather the 1,2,3-trione and the 1,2-iodoformylated products, further suggesting that any formation of α-iododiketone and α,α-diiododiketone intermediates would lead to undesired products rather than cyclopropanation (FIG. 12, panels G, H). These experiments confirm that α-iodo and α,α-diiodocarbonyls are neither substrates nor reaction intermediates in the current cyclopropanation and that the reaction most likely operates by a photoexcited O₂-mediated, PC*/I₂-catalyzed pathway in which O₂ enables both the generation of malonate α-C radicals and interception of secondary C-radicals to mediate ring closure.

The methods and compositions of the appended claims are not limited in scope by the specific methods and compositions described herein, which are intended as illustrations of a few aspects of the claims and any methods and compositions that are functionally equivalent are within the scope of this disclosure. Various modifications of the methods and compositions in addition to those shown and described herein are intended to fall within the scope of the appended claims. Further, while only certain representative methods, compositions, and aspects of these methods and compositions are specifically described, other methods and compositions and combinations of various features of the methods and compositions are intended to fall within the scope of the appended claims, even if not specifically recited. Thus, a combination of steps, elements, components, or constituents can be explicitly mentioned herein; however, all other combinations of steps, elements, components, and constituents are included, even though not explicitly stated.

### REFERENCES

[1] Davies, H. M. L. & Denton, J. R. Application of donor/acceptor-carbenoids to the synthesis of natural products. Chem. Soc. Rev. 38, 3061-3071 (2009).
[2] Carson, C. A. & Kerr, M. A. Heterocycles from cyclopropanes: applications in natural product synthesis. Chem. Soc. Rev. 38, 3051-3060 (2009).
[3] Zhang, D., Song, H. & Qin, Y. Total Synthesis of Indoline Alkaloids: A Cyclopropanation Strategy. Acc. Chem. Res. 44, 447-457 (2011).
[4] Talele, T. T. The “Cyclopropyl Fragment” is a Versatile Player that Frequently Appears in Preclinical/Clinical Drug Molecules. J. Med. Chem. 59, 8712-8756 (2016).
[5] Salaün, J. in Small Ring Compounds in Organic Synthesis VI (ed Armin de Meijere) 1-67 (Springer Berlin Heidelberg, 2000).
[6] Ebner, C. & Carreira, E. M. Cyclopropanation Strategies in Recent Total Syntheses. Chem. Rev. 117, 11651-11679 (2017).
[7] Donaldson, W. A. Synthesis of cyclopropane containing natural products. Tetrahedron 57, 8589-8627 (2001).
[8] Lebel, H., Marcoux, J.-F., Molinaro, C. & Charette, A. B. Stereoselective Cyclopropanation Reactions. Chem. Rev. 103, 977-1050 (2003).
[9] Doyle, M. P. in Modern Rhodium-Catalyzed Organic Reactions 341-355 (2005).
[10] Davies, H. M. L. & Antoulinakis, E. G. in Organic Reactions 1-326.
[11] Rancourt, J. et al. Peptide-Based Inhibitors of the Hepatitis C Virus NS3 Protease: Structure—Activity Relationship at the C-Terminal Position. J. Med. Chem. 47, 2511-2522 (2004).
[12] Caló, F. P., Zimmer, A., Bistoni, G. & Fürstner, A. From Serendipity to Rational Design: Heteroleptic Dirhodium Amidate Complexes for Diastereodivergent Asymmetric Cyclopropanation. J. Am. Chem. Soc. 144, 7465-7478 (2022).
[13] Green, S. P. et al. Thermal Stability and Explosive Hazard Assessment of Diazo Compounds and Diazo Transfer Reagents. Org. Process Res. Dev. 24, 67-84 (2020).
[14] Lathrop, S. P. et al. Continuous Process to Safely Manufacture an Aryldiazoacetate and Its Direct Use in a Dirhodium-Catalyzed Enantioselective Cyclopropanation. Org. Process Res. Dev. 27, 90-104 (2023).
[15] Morandi, B. & Carreira, E. M. Iron-Catalyzed Cyclopropanation in 6 M KOH with in Situ Generation of Diazomethane. Science 335, 1471-1474 (2012).
[16] Zhu, S., Perman, J. A. & Zhang, X. P. Acceptor/Acceptor-Substituted Diazo Reagents for Carbene Transfers: Cobalt-Catalyzed Asymmetric Z-Cyclopropanation of Alkenes with α-Nitrodiazoacetates. Angew. Chem. Int. Ed. 47, 8460-8463 (2008).
[17] Chen, Z.-L. et al. Enabling Cyclopropanation Reactions of Imidazole Heterocycles via Chemoselective Photochemical Carbene Transfer Reactions of NHC-Boranes. Org. Lett. 24, 2232-2237 (2022).
[18] Richter, M. J. R., Zécri, F. J., Briner, K. & Schreiber, S. L. Modular Synthesis of Cyclopropane-Fused N-Heterocycles Enabled by Underexplored Diazo Reagents. Angew. Chem. Int. Ed. 61, e202203221 (2022).
[19] Bordeaux, M., Tyagi, V. & Fasan, R. Highly Diastereoselective and Enantioselective Olefin Cyclopropanation Using Engineered Myoglobin-Based Catalysts. Angew. Chem. Int. Ed. 54, 1744-1748 (2015).
[20] Nam, D., Steck, V., Potenzino, R. J. & Fasan, R. A Diverse Library of Chiral Cyclopropane Scaffolds via Chemoenzymatic Assembly and Diversification of Cyclopropyl Ketones. J. Am. Chem. Soc. 143, 2221-2231 (2021).
[21] Wittmann, B. J. et al. Diversity-Oriented Enzymatic Synthesis of Cyclopropane Building Blocks. ACS Catal. 10, 7112-7116 (2020).
[22] Coelho, P. S., Brustad, E. M., Kannan, A. & Arnold, F. H. Olefin Cyclopropanation via Carbene Transfer Catalyzed by Engineered Cytochrome P450 Enzymes. Science 339, 307-310 (2013).
[23] Charette, A. B. & Beauchemin, A. in Org. React. 1-415 (2001).
[24] Jing, C., Jones, B. T., Adams, R. J. & Bower, J. F. Cyclopropane-Fused N-Heterocycles via Aza-Heck-Triggered C (sp3)-H Functionalization Cascades. J. Am. Chem. Soc. 144, 16749-16754 (2022).
[25] Sakurai, S., Inagaki, T., Kodama, T., Yamanaka, M. & Tobisu, M. Palladium-Catalyzed Siloxycyclopropanation of Alkenes Using Acylsilanes. J. Am. Chem. Soc. 144, 1099-1105 (2022).
[26] Zhang, L., DeMuynck, B. M., Paneque, A. N., Rutherford, J. E. & Nagib, D. A. Carbene reactivity from alkyl and aryl aldehydes. Science 377, 649-654 (2022).
[27] Phelan, J. P. et al. Redox-Neutral Photocatalytic Cyclopropanation via Radical/Polar Crossover. J. Am. Chem. Soc. 140, 8037-8047 (2018).
[28] Xu, B. et al. Photocatalyzed Diastereoselective Isomerization of Cinnamyl Chlorides to Cyclopropanes. J. Am. Chem. Soc. 142, 6206-6215 (2020).
[29] Li, A.-H., Dai, L.-X. & Aggarwal, V. K. Asymmetric Ylide Reactions: Epoxidation, Cyclopropanation, Aziridination, Olefination, and Rearrangement. Chem. Rev. 97, 2341-2372 (1997).
[30] Johnson, J. D., Teeples, C. R., Akkawi, N. R. & Wilkerson-Hill, S. M. Efficient Synthesis of Orphaned Cyclopropanes Using Sulfones as Carbene Equivalents. J. Am. Chem. Soc. 144, 14471-14476 (2022).
[31] Liu, M.-S., Du, H.-W., Cui, J.-F. & Shu, W. Intermolecular Metal-Free Cyclopropanation and Aziridination of Alkenes with XH2 (X=N, C) by Thianthrenation. Angew. Chem. Int. Ed. 61, e202209929 (2022).
[32] Kulinkovich, O. G. & de Meijere, A. 1,n-Dicarbanionic Titanium Intermediates from Monocarbanionic Organometallics and Their Application in Organic Synthesis. Chem. Rev. 100, 2789-2834 (2000).
[33] Corey, E. J., Rao, S. A. & Noe, M. C. Catalytic Diastereoselective Synthesis of Cis-1,2-Disubstituted Cyclopropanols from Esters Using a Vicinal Dicarbanion Equivalent. J. Am. Chem. Soc. 116, 9345-9346 (1994).
[34] Ni, J., Xia, X., Zheng, W.-F. & Wang, Z. Ti-Catalyzed Diastereoselective Cyclopropanation of Carboxylic Derivatives with Terminal Olefins. J. Am. Chem. Soc. 144, 7889-7900 (2022).
[35] Coscia, R. W. & Lambert, T. H. Development of a Formal [4+1] Cycloaddition: Pd(OAc)2-Catalyzed Intramolecular Cyclopropanation of 1,3-Dienyl β-Keto Esters and MgI2-Promoted Vinylcyclopropane-Cyclopentene Rearrangement. J. Am. Chem. Soc. 131, 2496-2498 (2009).
[36] Oumar-Mahamat, H., Moustrou, C., Surzur, J. M. & Bertrand, M. P. Lactone synthesis by manganese (III)-mediated oxidative cyclization of allylic .beta.-diesters. J. Org. Chem. 54, 5684-5688 (1989).
[37] Yang, D., Gao, Q., Lee, C.-S. & Cheung, K.-K. Novel Intramolecular Cyclopropanation Reaction of Unsaturated β-Keto Esters. Org. Lett. 4, 3271-3274 (2002).
[38] Jie, L.-H., Guo, B., Song, J. & Xu, H.-C. Organoelectrocatalysis Enables Direct Cyclopropanation of Methylene Compounds. J. Am. Chem. Soc. 144, 2343-2350 (2022).
[39] Fischer, D. M., Lindner, H., Amberg, W. M. & Carreira, E. M. Intermolecular Organophotocatalytic Cyclopropanation of Unactivated Olefins. J. Am. Chem. Soc. 145, 774-780 (2023).
[40] Prier, C. K., Rankic, D. A. & MacMillan, D. W. C. Visible Light Photoredox Catalysis with Transition Metal Complexes: Applications in Organic Synthesis. Chem. Rev. 113, 5322-5363 (2013).
[41] Narayanam, J. M. R. & Stephenson, C. R. J. Visible light photoredox catalysis: applications in organic synthesis. Chem. Soc. Rev. 40, 102-113 (2011).
[42] Fabry, D. C. & Rueping, M. Merging Visible Light Photoredox Catalysis with Metal Catalyzed C—H Activations: On the Role of Oxygen and Superoxide Ions as Oxidants. Acc. Chem. Res. 49, 1969-1979 (2016).
[43] Schmitz, G. The oxidation of iodine to iodate by hydrogen peroxide. Phys. Chem. Chem. Phys. 3, 4741-4746 (2001).
[44] Zhang, P., Le, C. C. & MacMillan, D. W. C. Silyl Radical Activation of Alkyl Halides in Metallaphotoredox Catalysis: A Unique Pathway for Cross-Electrophile Coupling. J. Am. Chem. Soc. 138, 8084-8087 (2016).
[45] Chen, Z.-L., Xie, Y. & Xuan, J. Visible Light-Mediated Cyclopropanation: Recent Progress. Eur. J. Org. Chem. 2022, e202201066 (2022).
[46] Wallentin, C.-J., Nguyen, J. D., Finkbeiner, P. & Stephenson, C. R. J. Visible Light-Mediated Atom Transfer Radical Addition via Oxidative and Reductive Quenching of Photocatalysts. J. Am. Chem. Soc. 134, 8875-8884 (2012).
[47] Ischay, M. A., Lu, Z. & Yoon, T. P. [2+2] Cycloadditions by Oxidative Visible Light Photocatalysis. J. Am. Chem. Soc. 132, 8572-8574 (2010).
[48] Ide, K., Furuta, M. & Tokuyama, H. Photoredox-catalyzed intramolecular cyclopropanation of alkenes with α-bromo-β-keto esters. Org. Biomol. Chem. 19, 9172-9176 (2021).
[49] Herraiz, A. G. & Suero, M. G. A transition-metal-free & diazo-free styrene cyclopropanation. Chem. Sci. 10, 9374-9379 (2019).
[50] Shiri, P., Ramezanpour, S., Amani, A. M. & Dehaen, W. A patent review on efficient strategies for the total synthesis of pazopanib, regorafenib and lenvatinib as novel anti-angiogenesis receptor tyrosine kinase inhibitors for cancer therapy. Mol. Divers. (2022).
[51] Cui, J. et al. Creation and manipulation of common functional groups en route to a skeletally diverse chemical library. Proc. Nat. Acad. Sci. 108, 6763-6768 (2011).
[52] Huang, S., Li, R., LaMontagne, K. R., Greenberger, L. M. & Connolly, P. J. 4-Aminopyrimidine-5-carbaldehyde oximes as potent VEGFR-2 inhibitors. Part II. Bioorg. Med. Chem. Lett. 21, 1815-1818 (2011).
[53] Iwaki, T. et al. Discovery and in vivo effects of novel human natriuretic peptide receptor A (NPR-A) agonists with improved activity for rat NPR-A. Bioorg. Med. Chem. 25, 6680-6694 (2017).
[54] Lee, W.-C. C. et al. Asymmetric radical cyclopropanation of dehydroaminocarboxylates: Stereoselective synthesis of cyclopropyl α-amino acids. Chem 7, 1588-1601 (2021).
[55] Yuen, T. Y., Brown, C. J., Tan, Y. S. & Johannes, C. W. Synthesis of Chiral Alkenyl Cyclopropane Amino Acids for Incorporation into Stapled Peptides. J. Org. Chem. 85, 1556-1566 (2020).
[56] Brackmann, F. & de Meijere, A. Natural Occurrence, Syntheses, and Applications of Cyclopropyl-Group-Containing α-Amino Acids. 1. 1-Aminocyclopropanecarboxylic Acid and Other 2,3-Methanoamino Acids. Chem. Rev. 107, 4493-4537 (2007).
[57] Zhang, D. et al. Efficient and Divergent Synthesis of Functionalized Cyclopropanes via Iodoform Reaction. Synthesis 44, 705-710 (2012).
[58] Schubert, H., Rack, M. & Mühlstädt, M. Eine vereinfachte Synthese der 1-Aminocyclopropan-1-carbonsäure. J. Prakt. Chem. 332, 812-814 (1990).
[59] Banerjee, R. K.; Dave, B.; Hangarge, R.; Wetal, R.; Palle, V. P.; Kamboj, R. K., Preparation of pyridopyrimidine compounds as MEK inhibitors for the treatment of diseases.
[60] Bertani, B.; Di Fabio, R.; Micheli, F.; Tedesco, G.; Terreni, S., Preparation of aza-bicyclic compounds as inhibitors of mono-amines re-uptake and antidepressant agents.
[61] Wu, W.-L.; Burnett, D. A.; Stamford, A.; Cumming, J. N.; Asberom, T.; Bennett, C.; Sasiskumar, T. K.; Scott, J. D., Preparation of spiroiminothiazine derivatives for use as BACE inhibitors.
[62] 1,1-Disubstituted alkenes generated Heck type products rather than cyclopropyl rings. Alkynes generate cyclopropenes in less than 10% yields.
[63] Han, C.; Cai, L.; Zhang, D.; Pan, R.; Li, Q.; Lin, A.; Yao, H. Site-Selective Palladium-Catalyzed 1,1-Arylamination of Terminal Alkenes. CCS Chem. 2022, 4, 616-624.
[64] Niehaus, J. C.; Hirtz, M.; Brinks, M. K.; Studer, A.; Fuchs, H.; Chi, L. Patterning of Functional Compounds by Multicomponent Langmuir-Blodgett Transfer and Subsequent Chemical Modification. Langmuir 2010, 26, 15388-15393.
[65] Gokel, G. W.; Hernandez, J. C.; Viscariello, A. M.; Arnold, K. A.; Campana, C. F.; Echegoyen, L.; Fronczek, F. R.; Candour, R. D.; Morgan, C. R.; Trafton, J. E.; Miller, S. R.; Minganti, C.; Eiband, D.; Schultz, R. A.; Tamminen, M. Number 14 Rouge, Louisiana 70803-1804; 1987; Vol. 52.
[66] Lo, H. K.; Luo, F. T. Synthesis of PS-Supported NHC-Pd Catalyst Derived from Theobromine and Its Applications in Suzuki-Miyaura Reaction. J. Chin. Chem. Soc. 2012, 59, 394-398.
[67] Fujita, T.; Yamamoto, T.; Morita, Y.; Chen, H.; Shimizu, Y.; Kanai, M. Chemo- and Enantioselective Pd/B Hybrid Catalysis for the Construction of Acyclic Quaternary Carbons: Migratory Allylation of O-Allyl Esters to α-C-Allyl Carboxylic Acids. J. Am. Chem. Soc. 2018, 140, 5899-5903.
[68] Guo, C.; Han, X.; Feng, Y.; Liu, Z.; Li, Y.; Liu, H.; Zhang, L.; Dong, Y.; Li, X. Straightforward Synthesis of Alkyl Fluorides via Visible-Light-Induced Hydromono- and Difluoroalkylations of Alkenes with α-Fluoro Carboxylic Acids. J. Org. Chem. 2022, 87, 9232-9241.
[69] Fatiadi, A. J. Novel Iodination of the A-Methylene Group in 1,3-DiphenylpropaneI 1,3-dione and Related F3-Diketones with Periodic Acid. J. Chem. Soc. D 1970, 11-11.
[70] Krasnokutskaya, E. A.; Lesina, Y. A.; Gorlushko, D. A.; Filimonov, V. D. Comparative Study of the Reactivity of Iodinating Agents in Solution and Solid Phase. 2005. Vol. 41.
[71] Huther, N.; McGrail, P. T.; Parsons, A. F. Radical Reactions Using Decacarbonyldimanganese under Biphasic Conditions. Eur. J. Org. Chem. 2004, 2004, 1740-1749.
[72] WO2016009306 A1
[73] WO2008031772 A1
[74] WO2013028670 A1

1. A method for forming a cyclopropyl or cyclopropenyl ring in a compound, the method comprising:
contacting an unsaturated hydrocarbon and an active methylene compound with an oxidant and a catalytically effective amount of a photocatalyst; and
irradiating the photocatalyst with a light source to form a cyclopropyl and/or cyclopropenyl-containing product.

2. The method of claim 1, wherein the unsaturated hydrocarbon comprises an alkene.

3. The method of claim 2, wherein the alkene comprises a terminal alkene.

4. The method of claim 3, wherein the terminal alkene comprises an alkene defined by the formula R¹(CH)CH₂,
wherein
R¹ is hydrogen, a halogen, a substituted or unsubstituted aryl, a substituted or unsubstituted alkyl, a substituted or unsubstituted alkylaryl, a substituted or unsubstituted heteroaryl, a substituted or unsubstituted alkylheteroaryl, —CHO, —CO₂R^{a}, —N(R^{a})₂, or —OR^{a}; and
each R^{a}, when present, is independently a substituted or unsubstituted aryl, a substituted or unsubstituted alkyl, a substituted or unsubstituted alkylaryl, a substituted or unsubstituted heteroaryl, or a substituted or unsubstituted alkylheteroaryl.

5. The method of claim 3, wherein the terminal alkene comprises 3-butenylbenzene.

6. The method of claim 2, wherein the alkene comprises an internal alkene.

7. The method of claim 1, wherein the unsaturated hydrocarbon comprises an alkyne.

8. The method of claim 1, wherein the active methylene compound is defined by the formula R² (CH₂) R³, wherein R² and R³ are each independently an electron withdrawing group.

9. The method of claim 8, wherein R² and R³ are each independently an electron withdrawing group selected from the group consisting of a halogen, —C(O)R^{b}, —COOR^{b}, —C(O)NR^{b}R^{c}, —CN, —SO₃R^{b}, —SO₂R^{b}, —SO₂NR^{b}R^{c}, —OP(O)OR^{b}OR^{c}, —R^{b}NCO, and —NO₂, wherein R^{b} and R^{c}, when present, are independently H or a substituted or unsubstituted C₁-C₆ alkyl.

10. The method of claim 1, wherein the active methylene compound is defined by the formula R^{b}OOC(CH₂)COOR^{c}, wherein R^{b} and R^{c} are independently H or a substituted or unsubstituted C₁-C₆ alkyl.

11. The method of claim 1, wherein the active methylene compound comprises diethylmalonate.

12. The method of claim 1, wherein the oxidant comprises a peroxide.

13. The method of claim 1, wherein the oxidant comprises an alkyl halide.

14. (canceled)

15. The method of claim 13, wherein the alkyl halide comprises iodocyclohexane.

16. The method of claim 1, wherein the oxidant comprises a first oxidant and a second oxidant, wherein the first oxidant comprises an alkyl halide, and wherein the second oxidant comprises O₂ or a peroxide.

17. (canceled)

18. (canceled)

19. (canceled)

20. (canceled)

21. The method of claim 1, wherein the photocatalyst comprises 4CzIPN, Eosin Y salts, [Ir(dtbbpy)(ppy)₂][BF₄], [Ir(dFCF₃ppy))₂(bpy)]PF₆, Ir(ppy)₃, or a combination thereof.

22. The method of claim 1, wherein the photocatalyst is irradiated with visible light.

23. The method of claim 22, wherein the visible light has a wavelength of from 390 nanometers (nm) to 467 nm.

24. The method of claim 1, wherein the unsaturated hydrocarbon, active methylene compound, oxidant, and photocatalyst are contacted in a solvent.

25. (canceled)

26. (canceled)

27. (canceled)

28. (canceled)

29. (canceled)

30. A method for forming a cyclopropyl or cyclopropenyl ring in a compound, the method comprising:
contacting an unsaturated hydrocarbon and an active methylene compound with an oxidant under conditions effective to form a cyclopropyl or cyclopropenyl ring.

31-56. (canceled)
